(19) United States
(12) Patent Application Publication
(10) Pub. No.: US 2018/0250261 A1
(43) Pub. Date: Sep. 6, 2018

(54) METHOD FOR TREATING CANCER WITH A STAT3 PATHWAY INHIBITOR AND KINASE INHIBITOR

(71) Applicant: BOSTON BIOMEDICAL, INC., Cambridge, MA (US)

(72) Inventors: Chiang J. LI, Cambridge, MA (US); Youzhi LI, Westwood, MA (US); Harry ROGOFF, Wrentham, MA (US); Yuan GAO, Belmont, MA (US); Janet HUANG, Watertown, MA (US)

(73) Assignee: BOSTON BIOMEDICAL, INC., Cambridge, MA (US)

(21) Appl. No.: 15/569,236

(22) PCT Filed: Apr. 26, 2016
(86) PCT No.: PCT/US2016/029328

§ 371 Date: Oct. 25, 2017

Related U.S. Application Data
(60) Provisional application No. 62/153,385, filed on Apr. 27, 2015.

(51) Int. Cl. A61K 31/343; A61K 31/427; A61P 35/02

(52) U.S. Cl. CPC A61K 31/343; A61K 2300/00; A61P 35/02; A61K 31/427

(57) ABSTRACT

Methods comprising administering and kits comprising at least one compound of formula A: (A) prodrugs, derivatives, pharmaceutically acceptable salts of any of the foregoing, and solvates of any of the foregoing, and at least one compound of formula B: (B) prodrugs, derivatives, pharmaceutically acceptable salts of any of the foregoing, and solvates of any of the foregoing.

The present application claims the benefit of priority under 35 U.S.C. § 119 of U.S. Provisional Patent Application No. 62/153,385, filed Apr. 27, 2015, the contents of which are incorporated herein by reference in its entirety.

Disclosed herein are methods comprising administering to a subject a combination comprising a therapeutically effective amount of at least one compound of formula A in combination with a therapeutically effective amount of at least one compound of formula B.

The at least one compound of formula A is chosen from compounds having formula A

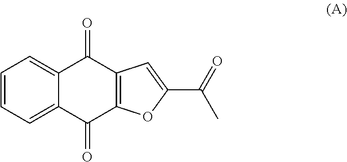 [SMILES: CC(=O)c1cc2c(o1)C(=O)c1ccccc1C2=O]

prodrugs, derivatives, pharmaceutically acceptable salts of any of the foregoing, and solvates of any of the foregoing.

The at least one compound of formula B is chosen from compounds having formula B

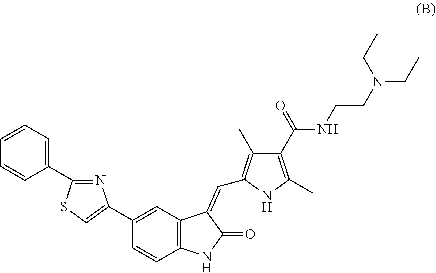 [SMILES: CCN(CC)CCNC(=O)c1c(C)[nH]c(/C=C2\C(=O)Nc3ccc(-c4csc(-c5ccccc5)n4)cc32)c1C]

prodrugs, derivatives, pharmaceutically acceptable salts of any of the foregoing, and solvates of any of the foregoing.

Cancer fatalities in the United States alone number in the hundreds of thousands each year. Despite advances in the treatment of certain forms of cancer through surgery, radiotherapy, and chemotherapy, many types of cancer are essentially incurable. Even when an effective treatment is available for a particular cancer, the side effects of such treatment can be severe and result in a significant decrease in quality of life.

Most conventional chemotherapy agents have toxicity and limited efficacy, particularly for patients with advanced solid tumors. Conventional chemotherapeutic agents cause damage to non-cancerous as well as cancerous cells. The therapeutic index of these chemotherapeutic compounds (i.e., a measure of the therapy's ability to distinguish between cancerous and normal cells) can be quite low. Frequently, a dose of a chemotherapy drug that is effective at killing cancer cells will also kill normal cells, especially those normal cells (such as epithelial cells and cells of the bone marrow) that undergo frequent cell division. When normal cells are subject to by chemotherapy, side effects such as hair loss, suppression of hematopoiesis, and nausea often occur. Depending on the general health of the patient, such side effects can preclude the administration of chemotherapy all together, or, at least, inflict significant discomfort on cancer patients that diminishes their quality of life. Even for cancer patients who respond to chemotherapy with tumor regression, cancers often quickly relapse, progress, and spread by metastasis after the initial response to chemotherapy. Such recurrent cancers become highly resistant or refractory to chemotherapeutics. As discussed below, cancer stem cells (CSCs) or cancer cells with high stemness (stemness-high cancer cells) are believed to be responsible for the rapid tumor recurrence and resistance observed after traditional chemotherapy.

CSCs are believed to possess the following four characteristics:

1. Stemness—As used herein, stemness means the capacity to self-renew and differentiate into cancer cells (Gupta P B et al., Nat. Med. 2009; 15(9):1010-1012). While CSCs are only a minor portion of the total cancer cell population (Clarke M F, Biol. Blood Marrow Transplant. 2009; 11(2 suppl 2):14-16), they can give rise to heterogeneous lineages of cancer cells that make up the bulk of the tumor (see Gupta et al. 2009). In addition, CSCs possess the ability to mobilize to distinct sites while retaining their stemness properties and thus regrowth of the tumor at these sites (Jordan C T et al. N. Engl. J. Med. 2006; 355(12):1253-1261).

2. Aberrant signaling pathways—CSC stemness is associated with dysregulation of signaling pathways, which may contribute to their ability to regrow tumors and to migrate to distant sites. In normal stem cells, stemness signaling pathways are tightly controlled and genetically intact. In contrast, stemness signaling pathways in CSCs are dysregulated, allowing these cells to self-renew and differentiate into cancer cells (see Ajani et al. 2015). Dysregulation of stemness signaling pathways contributes to CSC resistance to chemotherapy and radiotherapy and to cancer recurrence and metastasis. Exemplary stemness signaling pathways involved in the induction and maintenance of stemness in CSCs include: JAK/STAT, Wnt/β-catenin, Hedgehog, Notch, and Nanog (Boman B M et al., J. Clin. Oncol. 2008; 26(17)2828-2838).

3. Resistance to traditional therapies—evidence suggests that CSCs possess resistance to conventional chemotherapy and radiation. While the detailed mechanism underlying such resistance is not well understood, the stemness pathways of CSCs (see Boman et al. 2008) together with the tumor microenvironment and aberrant regulation of signaling pathways (Borovski T. et al., Cancer Res. 2011; 71(3):634-639) may contribute to such resistance.

4. Ability to contribute to tumor recurrence and metastasis—although chemotherapy and radiation may kill most of the cells in a tumor, since CSCs are resistant to traditional therapies, the CSCs that are not eradicated may lead to regrowth or recurrence of the tumor either at the primary site or at distant sites (see Jordan et al. 2006). As mentioned above, CSCs may acquire the ability to mobilize to different sites and may maintain stemness at these sites through interactions with the microenvironment, allowing for metastatic tumor growth (see Boman et al. 2008).

The transcription factor Signal Transducer and Activator of Transcription 3 (referred to herein as STAT3) is a member of the STAT family, which are latent transcription factors activated in response to cytokines/growth factors to promote proliferation, survival, and other biological processes. STAT3 is an oncogene that can be activated by phosphorylation of a critical tyrosine residue mediated by growth factor receptor tyrosine kinases, including but not limited to, e.g., Janus kinases (JAKs), SRC family kinases, EGFR, ABL, KDR, c-MET, and HER2. (Yu, H. Stat3: Linking oncogenesis with tumor immune evasion in AACR 2008 Annual Meeting. 2008, San Diego, Calif.). Upon tyrosine phosphorylation, the phosphorylated STAT3 (“pSTAT3”) forms homo-dimers and translocates to the nucleus, where it binds to specific DNA-response elements in the promoters of target genes, and induces gene expression. (Pedranzini, L., et al. J. Clin. Invest., 2004. 114(5): p. 619-22).

In normal cells, STAT3 activation is transient and tightly regulated, lasting for example from about 30 minutes to several hours. However, in a wide variety of human cancers, including all the major carcinomas as well as some hematologic tumors, STAT3 is found to be aberrantly active. Persistently active STAT3 occurs in more than half of all breast and lung cancers, colorectal cancers (CRC), ovarian cancers, hepatocellular carcinomas, and multiple myelomas, etc., and in more than 95% of all head/neck cancers. STAT3 plays multiple roles in cancer progression and is considered to be one of the principal mechanisms by which cancer cells acquire drug resistance. STAT3 is a potent transcription regulator that targets genes involved in cell cycle, cell survival, oncogenesis, tumor invasion, and metastasis, such as BCL-XL, c-MYC, CYCLIN D1, VEGF, MMP-2, and SURVIVIN. (Catlett-Falcone, R., et al. Immunity, 1999. 10(1): p. 105-15; Bromberg, J. F., et al. Cell, 1999. 98(3): p. 295-303; Kanda, N., et al. Oncogene, 2004. 23(28): p. 4921-29; Schlette, E. J., et al. J Clin Oncol, 2004. 22(9): p. 1682-88; Niu, G., et al. Oncogene, 2002. 21(13): p. 2000-08; Xie, T. X., et al. Oncogene, 2004. 23(20): p. 3550-60). STAT3 is also a key negative regulator of tumor immune surveillance and immune cell recruitment. (Kortylewski, M., et al. Nat. Med., 2005. 11(12): p. 1314-21; Burdelya, L., et al. J. Immunol., 2005. 174(7): p. 3925-31; and Wang, T., et al. Nat. Med., 2004. 10(1): p. 48-54).

Abrogation of STAT3 signaling by using anti-sense oligonucleotides, siRNA, dominant-negative form of STAT3, and/or the targeted inhibition of tyrosine kinase activity results in cancer cell-growth arrest, apoptosis, and reduction of metastasis frequency both in vitro and/or in vivo, (Pedranzini, L., et al. J Clin. Invest., 2004. 114(5): p. 619-22; Bromberg, J. F., et al. Cell, 1999. 98(3): p. 295-303; Darnell, J. E. Nat. Med., 2005. 11(6): p. 595-96; and Zhang, L., et al. Cancer Res, 2007. 67(12): p. 5859-64).

Furthermore, STAT3 may play a key role in the survival and self-renewal capacity of CSCs across a broad spectrum of cancers. Therefore, an agent with activity against CSCs holds great promise for cancer patients (Boman, B. M., et al. J. Clin. Oncol. 2008. 26(17): p. 2795-99).

As discussed above, CSCs are a sub-population of cancer cells (found within solid tumors or hematological cancers) that possess characteristics normally associated with stem cells. These cells grow faster after reduction of non-stem regular cancer cells by chemotherapy, which may provide a mechanism by which cancers are able to relapse quickly after chemotherapy treatment. In contrast to the bulk of cancer cells, CSCs are highly tumorigenic (tumor-forming). In human acute myeloid leukemia, the frequency of these cells is less than 1 in 10,000, (Bonnet, D. and J. E. Dick. Nat. Med., 1997. 3(7): p. 730-37). There is mounting evidence that such cells exist in almost all tumor types. However, cancer cell lines that are selected from a sub-population of cancer cells that are specifically adapted to growth in tissue culture, may acquire biological and functional properties that differ significantly from cancer cells in vivo. Thus, not all cancer cell lines contain CSCs.

CSCs have stem cell properties such as self-renewal and the ability to differentiate into multiple cell types. They persist in tumors as a distinct population and they give rise to the differentiated cells that form the bulk of the tumor mass and phenotypically characterize the disease, CSCs have been demonstrated to be fundamentally responsible for carcinogenesis, cancer metastasis, cancer recurrence, and relapse. CSCs are also called, for example, tumor initiating cells, cancer stem-like cells, stem-like cancer cells, highly tumorigenic cells, or super malignant cells.

CSCs are inherently resistant to conventional chemotherapies, which means they survive conventional therapies that kill the bulk of tumor cells. As such, the existence of CSCs has several implications in terms of cancer treatment and therapy. These include, for example, disease identification, selective drug targets, prevention of cancer metastasis and recurrence, treatment of cancer refractory to chemotherapy and/or radiotherapy, treatment of cancers inherently resistant to chemotherapy or radiotherapy and development of new strategies in fighting cancer.

The efficacy of cancer treatments are, in the initial stages of testing, often measured by the amount of tumor mass they kill off. Because CSCs form a minor proportion of the tumor cell population and have markedly different biologic characteristics than their differentiated progeny, the selection of treatment regimens based on their ability to reduce tumor mass may not select for drugs that act specifically on stem cells. In fact, CSCs are radio-resistant and refractory to chemotherapeutic and targeted drugs. Normal somatic stem cells are naturally resistant to chemotherapeutic agents—they have various pumps (e.g., multidrug resistance protein pump) that efflux drugs, they have a higher DNA repair capability, and they have a slow rate of cell turnover. CSCs, being the mutated counterparts of normal stem cells, may have similar functions. For example, CSCs may evade cell death induced by standard chemotherapy because chemotherapeutic agents target primarily rapidly replicating cells that form the bulk of the tumor. Thus, it is the survival of the CSC population resident in the tumor that ultimately leads to a relapse of the disease and widespread metastasis. Treatment with chemotherapeutic agents may in fact select for chemotherapy-resistant CSCs that are able to seed tumors that are most likely to be resistant to chemotherapy. Moreover, cancer stem cells have also been demonstrated to be resistant to radiation therapy (XRT). (Hambardzumyan, et al. Cancer Cell, 2006. 10(6): p. 454-56; and Baumann, M., et al. Nat. Rev. Cancer, 2008. 8(7): p. 545-54).

Because the survival of CSCs may be the principal cause for relapse, anti-cancer therapies that specifically target CSCs hold great promise. (Jones R J et al., J Natl Cancer Inst. 2004; 96(8):583-585). By targeting CSC pathways, it may be possible to treat patients with aggressive, non-resectable tumors and refractory or recurrent cancers as well as prevent tumor metastasis and recurrence. Such approach may also improve the survival and quality of life of cancer patients, especially those patients suffering from metastatic disease. Unlocking this untapped potential may involve the identification and validation of pathways that are selectively important for CSC self-renewal and survival. While signalling pathways regulating embryonic or adult stem cell proliferation and differentiation are well known, it remains to be seen if these same pathways are required for cancer stem cell self-renewal and survival.

Methods for identification and isolation of CSCs rely on the ability of CSCs to efflux drugs or express specific cell surface markers.

For example, because CSCs are resistant to many chemotherapeutic agents, it is not surprising that CSCs almost ubiquitously overexpress drug efflux pumps such as ABCG2 (BCRP-1), and other ATP binding cassette (ABC) superfamily members. (Ho, M. M., et al. Cancer Res., 2007. 67(10): p. 4827-33; Wang, J., et al. Cancer Res., 2007. 67(8): p. 3716-24; Haraguchi, N., et al. Stem Cells, 2006. 24(3): p. 506-13; Doyle, L. A. and D. D. Ross. Oncogene, 2003. 22(47): p. 7340-58; Alvi, A. J., et al. Breast Cancer Res., 2003. 5(1): p. R1-R8; Frank, N. Y., et al. Cancer Res., 2005. 65(10): p. 4320-33; and Schatton, T., et al. Nature, 2008. 451(7176): p. 345-49). Accordingly, the side population (SP) technique, originally used to enrich hematopoietic and leukemic stem cells, has also been employed to identify and isolate CSCs. (Kondo, T., et al. Proc. Natl Acad. Sci. USA, 2004. 101(3): p. 781-86). This technique, first described by Goodell et al., takes advantage of differential ABC transporter-dependent efflux of fluorescent dyes, such as Hoechst 33342, in order to define a cell population enriched in CSCs, (Doyle, L. A, and D. D. Ross, Oncogene, 2003. 22(47): p. 7340-58; and Goodell, M. A., et al. J. Exp. Med., 1996. 183(4): p. 1797-806). Specifically, the SP is identified by blocking drug efflux with verapamil, at which point the dyes can no longer be pumped out of the SP.

Efforts have also focused on finding specific markers that distinguish CSCs from the bulk of the tumor cells. Markers originally associated with normal adult stem cells have been found to also mark CSCs and co-segregate with the enhanced tumorigenicity of CSCs. Surface markers commonly expressed by the CSCs include CD44, CD133, and CD166. (Al-Hajj, M., et al. Proc. Natl Acad. Sci. USA, 2003. 100(7): p. 3983-88; Collins, A. T., et al. Cancer Res., 2005. 65(23): p. 10946-51; Li, C., et al. Cancer Res., 2007. 67(3): p. 1030-37; Ma, S., et al. Gastroenterology, 2007. 132(7): p. 2542-56; Ricci-Vitiani, L., et al. Nature, 2007. 445(7123): p. 111-15; Singh, S. K., et al. Cancer Res., 2003. 63(18): p. 5821-28; and Bleau, A. M., et al., Neurosurg. Focus, 2008. 24(3-4): p. E28). Sorting tumor cells based primarily upon the differential expression of these surface marker(s) have accounted for the majority of the highly tumorigenic CSCs described to date. Therefore, these surface markers are validated for identification and isolation of CSCs from the cancer cell lines and from the bulk of tumor tissues.

Protein kinases are a family of enzymes that regulate a wide variety of cellular processes, including cell growth, cell proliferation, cell differentiation, and metabolism. Protein kinases communicate cell growth signals through sequential chemical modification of pathway partners. Therefore, pharmacologic inhibition of any kinase on a given signal transduction cascade would theoretically block communication along the entire pathway. In addition, it is known that protein kinases play a role in disease states and disorders, for example, kinase mutation and/or overexpression are frequently present in many cancers, resulting in hyper-activated activity that often correlates with uncontrolled cell growth. For that reason, protein kinases represent potential targets for therapeutic inhibition.

As disclosed in U.S. Pat. No. 8,299,106, kinases have recently been shown to be important targets for killing or inhibiting cancer stem cells and collectively referred to as cancer stem cell pathway kinases (CSCPK). Non-limiting examples of CSCPKs include STK33, MELK, AXL, p70S6K, and PDGFRα. For example, PDGFRα is a receptor tyrosine kinase (RTK) that is activated after binding to its ligand, PDGF, and thereby contributes to cell proliferation, angiogenesis, and apoptosis. PDGFRα, belongs to the class III receptor tyrosine kinase family and it is related to the CFS-1 receptor/c-fms and the stem cell growth factor/c-kit proto-oncogene family. The PDGFRα pathway which is active in early fetal development is also reactivated in many cancers, such as hepatocellular cancer (HCC), head and neck cancer, brain tumors, gastrointestinal tumors, skin cancer, prostate cancer, ovarian cancer, breast cancer, sarcoma, and leukemia. In addition, PDGFRα activation has recently been shown to play a key role in bone metastasis of prostate cancer. The PDGFRα-p70S6K pathway is also essential for angiogenesis in vivo. Specifically targeting PDGFRα using a monoclonal antibody leads to significant reduction in tumor cell proliferation and survival with minimal toxicity. Therefore, PDGFRα represents a key target for developing therapies against a broad spectrum of cancers with minimal toxicity.

Other than cancer, chromosomal rearrangements are also known to activate PDGFRα by fusion to FIP1L1, which causes idiopathic hypereosinophilic syndrome. In addition, activation of PDGFRα by promoter polymorphisms has been linked to neural tube defects, such as spina bifida. PDGFRα activation has also been linked with fibrosis. Thus, PDGFRα also represents a potential target for anti-fibrotic therapy.

In some embodiments, the at least one compound of formula A is an inhibitor of CSC growth and survival. According to U.S. Pat. No. 8,877,803, the compound of formula A is shown to inhibit STAT3 pathway activity with a cellular IC50 of ˜0.25 μM. U.S. Pat. No. 8,877,803, for example, Example 13 further provides exemplary methods of synthesizing at least one compound of formula A. In some embodiments, the at least one compound of formula A is used in a method of treating cancers. For example, in PCT Patent Application No. PCT/US2014/033566, Example 6, the at east one compound of formula A was chosen to enter a clinical trial for patients with advanced cancers. The disclosures of U.S. Pat. No. 8,877,803 and PCT Patent Application No. PCT/US2014/033566 are incorporated herein by reference in their entireties.

In some embodiments, the at least one compound of formula B is an inhibitor of a CSCPK. As disclosed in U.S. Pat. No. 8,299,106, the compounds of formula B inhibit CSC. Examples 1-5 of U.S. Pat. No. 8,299,106 further provide exemplary methods of synthesizing the at least one compound of formula B. The disclosures of U.S. Pat. No. 8,299,106 are incorporated herein by reference in their entireties.

The present disclosure reports on the surprising discovery that a treatment combination of at least one compound of formula A and at least one compound of formula B had a greater effect in inhibiting cancer cells, including cancer stem cells, than the added effects of both compounds alone. For example, enhanced inhibition of the expression of cancer cell stemness-associated factors in vitro and in vivo, as well as cancer stem cells in vitro and in vivo, by a treatment combination of the present disclosure compared to the treatment with Compound A or Compound B alone were observed.

For example, we surprisingly discovered that a treatment combination of human pancreatic (Panc-1) cancer cells and human head and neck (FaDu) cancer cells with Compound A and Compound B resulted in enhanced inhibition of phospho-STAT3 expression when compared to the treatment with Compound A or Compound B alone. For example, we surprisingly discovered that a treatment combination of human gastric (MKN28) cancer cells with Compound A and Compound B resulted in enhanced inhibition of Nanog expression compared to the treatment with Compound A or Compound B alone. For example, we surprisingly discovered that administration of a treatment combination of Compound A and Compound B resulted in enhanced knockdown of the cancer cell stemness-associated factors Nanog and STK33 and in human colon (SW480) cancer xenograft tissue compared to the treatment with Compound A or Compound B alone.

For example, we surprisingly discovered that the administration of a treatment combination of Compound A and Compound B resulted in enhanced inhibition of CSC sphere formation in vitro when compared to the treatment with Compound A or Compound B alone. For example, we surprisingly discovered that a treatment combination of mice harboring human colon (SW480) xenograft tumors with Compound A and Compound B resulted in enhanced in vivo anti-CSC activity.

For example, we observed enhanced anticancer activity following treatment of mice harboring various human xenograft tumors with a treatment combination of Compound A and Compound B compared to the treatment with Compound A or Compound B alone. In a human colon cancer (SW480) xenograft model, a treatment combination with Compound A and Compound B enhanced tumor growth inhibition compared to Compound A or Compound B used alone at subtherapeutic doses, with tumor growth inhibition calculated to be 77% for the Compound A+Compound B combination (p<0.0005). We have observed similar results with a human gastric cancer (MKN45) xenograft model (69% tumor growth inhibition; p<0.0121) following a treatment combination with Compound A and Compound B.

Without being limited to any particular observation or hypothesis, the components of a treatment combination of the present disclosure are believed to work on different pathways that are associated with cancer cells (e.g., CSC). The treatment combination of a Compound A and a Compound B itself exerts effects that are greater than the additive effects of the two compounds alone (sometimes referred to as “enhanced” or “synergistic” effects). As illustrated in FIG. 1, Compound A can work by inhibiting the STAT3 signalling pathway. Specifically, Compound A can directly bind and inhibit the activity of activated STAT3 (e.g., phosphorylated STAT3), thereby preventing transcription of STAT3-dependent target genes including the stemness-associated transcription factors c-MYC, OCT4 SOX2, and β-CATENIN. In contrast to Compound A, which can block STAT3 activities via a kinase independent mechanism, Compound B can inhibit the activity of multiple malignancy-associated serine-threonine kinases (or cancer stem cell pathway kinases (CSCPKs)). As further illustrated in FIG. 1, blockade of CSCPK by Compound B can also lead to the down regulation of various cancer cell stemness-associated factors including Nanog. As discussed herein, effects greater than the additive effects of Compound A or Compound B alone were observed when cancer cells were treated with a treatment combination of a Compound A and a Compound B.

In some embodiments, disclosed herein are methods for treating cancer comprising administering to a subject in need thereof:
a therapeutically effective amount of at least one compound of formula A chosen from compounds having formula A:

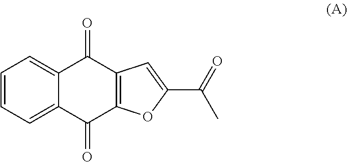 [SMILES: CC(=O)c1cc2c(o1)C(=O)c1ccccc1C2=O]

prodrugs, derivatives, pharmaceutically acceptable salts of any of the foregoing, and solvates of any of the foregoing, and
a therapeutically effective amount of at least one compound of formula B chosen from compounds having formula B:

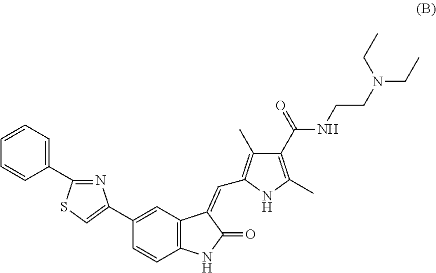 [SMILES: CCN(CC)CCNC(=O)c1c(C)[nH]c(/C=C2\C(=O)Nc3ccc(-c4csc(-c5ccccc5)n4)cc32)c1C]

prodrugs, derivatives, pharmaceutically acceptable salts of any of the foregoing, and solvates of any of the foregoing.

In some embodiments, disclosed herein are methods of treating cancer comprising administering to a subject in need thereof (a) a therapeutically effective amount of a cancer stemness inhibitor, a prodrug of the foregoing, a derivative of the foregoing, a pharmaceutically acceptable salt of any of the foregoing, or a solvate of any of the foregoing; and (b) a therapeutically effective amount of a kinase-targeting agent, a prodrug of the foregoing, a derivative of the foregoing, a pharmaceutically acceptable salt of any of the foregoing, or a solvate of any of the foregoing. In some embodiments, the cancer stemness inhibitor is a STAT3 pathway inhibitor.

In some embodiments, the cancer stemness inhibitor is chosen from 2-(1-hydroxyethyl)-naphtho[2,3-b]furan-4,9-dione, 2-acetyl-7-chloro-naphtho[2,3-b]furan-4,9-dione, 2-acetyl-7-fluoro-naphtho[2,3-b]furan-4,9-dione, 2-acetylnaphtho[2,3-b]furan-4,9-dione, 2-ethyl-naphtho[2,3-b]furan-4,9-dione, prodrugs of any of the foregoing, derivatives of any of the foregoing, pharmaceutically acceptable salts of any of the foregoing, and solvates of any of the foregoing.

In some embodiments, the kinase-targeting agent is a kinase inhibitor. In some embodiments, the kinase-targeting agent is a cancer stem cell pathway kinase inhibitor. In some embodiments, the kinase-targeting agent is chosen from

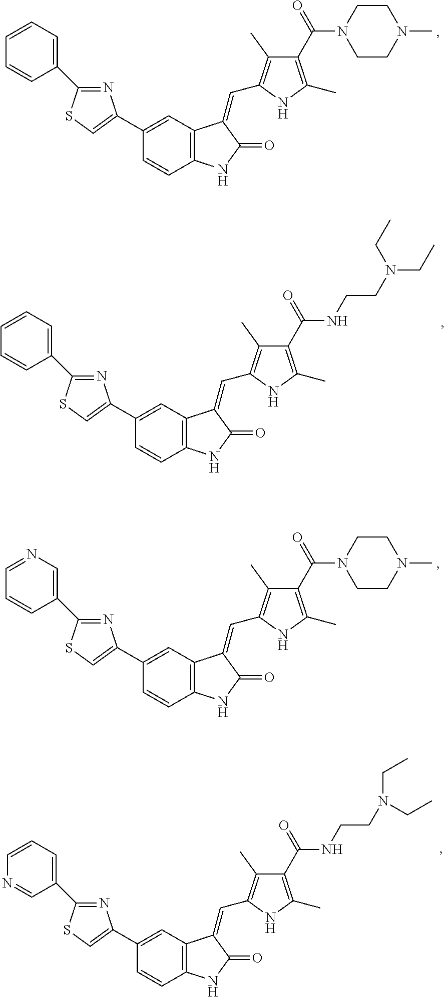 [SMILES: Cc1[nH]c(/C=C2\C(=O)Nc3ccc(-c4csc(-c5ccccc5)n4)cc32)c(C)c1C(=O)N1CCN(C)CC1 ; CCN(CC)CCNC(=O)c1c(C)[nH]c(/C=C2\C(=O)Nc3ccc(-c4csc(-c5ccccc5)n4)cc32)c1C ; Cc1[nH]c(/C=C2\C(=O)Nc3ccc(-c4csc(-c5cccnc5)n4)cc32)c(C)c1C(=O)N1CCN(C)CC1 ; CCN(CC)CCNC(=O)c1c(C)[nH]c(/C=C2\C(=O)Nc3ccc(-c4csc(-c5cccnc5)n4)cc32)c1C]

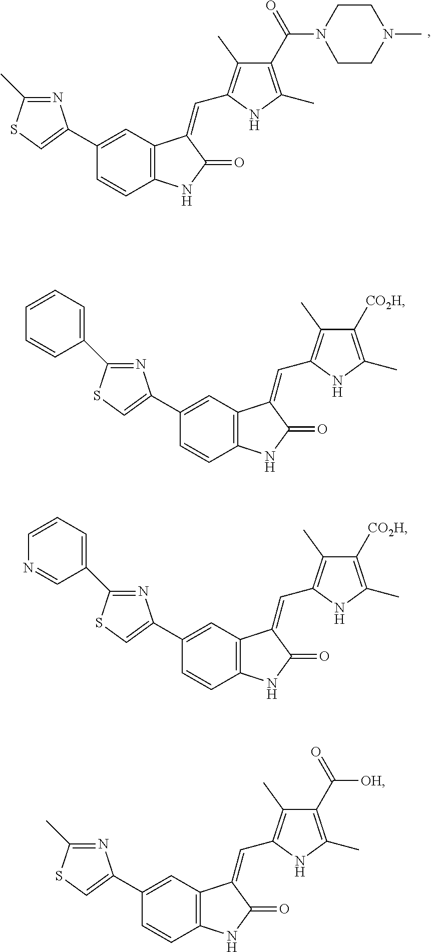

prodrugs of any of the foregoing, derivatives of any of the foregoing, pharmaceutically acceptable salts of any of the foregoing, and solvates of any of the foregoing.

In some embodiments, a kit is disclosed that comprises (1) at least one compound chosen from compounds having formula A, prodrugs, derivatives, pharmaceutically acceptable salts of any of the foregoing, and solvates of any of the foregoing, and (2) at least one compound chosen from compounds having formula B, prodrugs, derivatives, pharmaceutically acceptable salts of any of the foregoing, and solvates of any of the foregoing, together with instructions for administration and/or use.

In some embodiments, a kit is disclosed that comprises (1) at least one cancer stemness inhibitor, a prodrug of the foregoing, a derivative of the foregoing, a pharmaceutically acceptable salt of any of the foregoing, or a solvate of any of the foregoing; and (2) at least one kinase-targeting agent, a prodrug of the foregoing, a derivative of the foregoing, a pharmaceutically acceptable salt of any of the foregoing, or a solvate of any of the foregoing, together with instructions for administration and/or use.

Aspects and embodiments of the present disclosure are set forth or will be readily apparent from the following detailed description. It is to be understood that both the foregoing general description and the following detailed description are exemplary and explanatory only, and are not intended to be restrictive of the claims.

### BRIEF DESCRIPTION OF THE DRAWINGS

FIG. 1 illustrates enhanced inhibition of cancer cell stemness by a treatment combination of Compound A and Compound B.

FIG. 2 shows an enhanced inhibition of STAT3 phosphorylation following a treatment combination of Compound A (“608”) and Compound B (“503”).

FIG. 3 shows an enhanced inhibition of Nanog protein expression following a treatment combination of Compound A and Compound B.

FIG. 4 shows enhanced inhibition of 786-0, RKO, and DLD-1 cell colony formation following a treatment combination of Compound A and Compound B.

FIG. 5 shows that a treatment combination of Compound A and Compound B resulted in an enhanced inhibition of cancer stem cell viability.

FIGS. 6(A)-(B) show enhanced knockdown in the protein expression of pharmacodynamic markers of Compound A and Compound B following a treatment combination of Compound A and Compound B.

FIG. 7 shows enhanced in vivo anti-cancer stem activity following a treatment combination of Compound A and Compound B.

FIG. 8 shows enhanced anti-tumor activity in a mouse xenograft model of human colon cancer following a treatment combination of Compound A and Compound B.

FIG. 9 shows enhanced anti-tumor activity in a mouse xenograft model of human gastric cancer following a treatment combination of Compound A and Compound B.

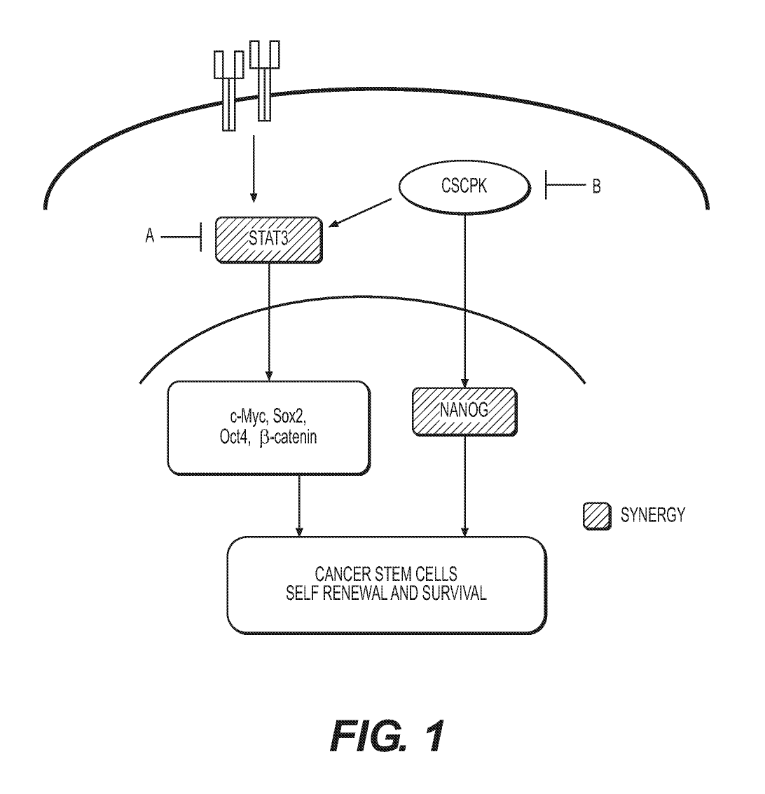

The following are definitions of terms used in the present specification. The initial definition provided for a group or term herein applies to that group or term throughout the present specification individually or as part of another group, unless otherwise indicated.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1% In some embodiments, the term “about” is used to modify a numerical value above and below the stated value by a variance of 10%. In some embodiments, the term “about” is used to modify a numerical value above and below the stated value by a variance of 5%. In some embodiments, the term “about” is used to modify a numerical value above and below the stated value by a variance of 1%.

The terms “administer,” “administering,” or “administration” are used herein in their broadest sense. These terms refer to any method of introducing to a subject a compound or pharmaceutical composition described herein and can include, for example, introducing the compound systemically, locally, or in situ to the subject. Thus, a compound of the present disclosure produced in a subject from a composition (whether or not it includes the compound) is encompassed by these terms. When these terms are used in connection with the term “systemic” or “systemically,” they generally refer to in vivo systemic absorption or accumulation of the compound or composition in the blood stream followed by distribution throughout the entire body.

The term “subject” generally refers to an organism to which a compound or pharmaceutical composition described herein can be administered. A subject can be a mammal or mammalian cell, including a human or human cell. The term also refers to an organism, which includes a cell or a donor or recipient of such cell. In various embodiments, the term “subject” refers to any animal (e.g., a mammal), including, but not limited to humans, mammals and non-mammals, such as non-human primates, mice, rabbits, sheep, dogs, cats, horses, cows, chickens, amphibians, reptiles, fish, nematode, and insects, which is to be the recipient of a compound or pharmaceutical composition described herein. Under some circumstances, the terms “subject” and “patient” are used interchangeably herein in reference to a human subject.

The terms “effective amount” and “therapeutically effective amount” refer to that amount of a compound or pharmaceutical composition described herein that is sufficient to effect the intended result including, but not limited to, disease treatment, as illustrated below. In some embodiments, the “therapeutically effective amount” is the amount that is effective for detectable killing or inhibition of the growth or spread of cancer cells, the size or number of tumors, and/or other measure of the level, stage, progression and/or severity of the cancer. In some embodiments, the “therapeutically effective amount” refers to the amount that is administered systemically, locally, or in situ (e.g., the amount of compound that is produced in situ in a subject). The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of cell migration. The specific dose may vary depending on, for example, the weight of the subject, the particular pharmaceutical composition, subject and their age and existing health conditions or risk for health conditions, the dosing regimen to be followed, the severity of the disease, whether it is administered in combination with other agents, timing of administration, the tissue to which it is administered, and the physical delivery system in which it is carried.

As used herein, the terms “treatment, ” “treating,” “ameliorating,” and “encouraging” are used interchangeably herein. These terms refer to an approach for obtaining beneficial or desired results including, but not limited to, a therapeutic benefit and/or prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject can still be afflicted with the underlying disorder. For prophylactic benefit, the pharmaceutical composition may be administered to a subject at risk of developing a particular disease, or to a subject reporting one or more of the physiological symptoms of a disease, even though a diagnosis of this disease may not have been made.

The terms “combination,” “combinatorial,” or “combination treatment,” as used herein, mean the administration of at least two different agents (e.g., at least one compound chosen from compounds having formula A or/and at least one compound chosen from compounds having formula B, as well as one or more additional agents) to treat a disorder, condition, or symptom, e.g., a cancer condition. Such combination/combination treatment may involve the administration of one agent before, during, and/or after the administration of a second agent. The first agent and the second agent can be administered concurrently, separately, or sequentially to a subject in separate pharmaceutical compositions. The first agent and the second agent may be administered to a subject by the same or different routes of administration. In some embodiments, a treatment combination comprises a therapeutically effective amount of at least one compound of formula A chosen from compounds having formula A and a therapeutically effective amount of at least one compound of formula B chosen from compounds having formula B.

For example, the at least one compound chosen from compounds having formula A and the at least one compound chosen from compounds having formula B can have different mechanisms of action. In some embodiments, a combination treatment improves the prophylactic or therapeutic effect of the at least one compound chosen from compounds having formula A and the at least one compound chosen from compounds having formula B by functioning together to have an additive, synergistic, or enhanced effect. In certain embodiments, a combination treatment of the present disclosure reduces the side effects associated with the at least one compound chosen from compounds having formula A or the at least one compound chosen from compounds having formula B. The administrations of the at least one compound chosen from compounds having formula A and the at least one compound chosen from compounds having formula B may be separated in time by up to several weeks, but more commonly within 48 hours, and most commonly within 24 hours.

The term “synergy,” “synergistic,” “synergistically,” or “enhanced” as used herein refers to an effect of interaction or combination of two or more components to produce a combined effect greater than the sum of their separate effects (or “additive effects”).

The terms “progress,” “progressed,” and “progression” as used herein refer to at least one of the following: (1) a response to prior therapy (e.g., chemotherapy) of progressive disease (PD); (2) the appearance of one or more new lesions after treatment with prior therapy (e.g., chemotherapy); and (3) at least a 5% (e.g., 10%, 20%) increase in the sum of diameters of target lesions, taking as a reference the smallest sum on study (this includes the baseline sum if that is the smallest on study).

As used herein, “sensitizing” means making subjects who were previously resistant, non-responsive, or somewhat responsive to a therapy (e.g., chemotherapy) regimen sensitive, responsive, or more responsive to that therapy (e.g., chemotherapy) regimen.

The term “cancer” in a subject refers to the presence of cells possessing characteristics typical of cancer-causing cells, such as uncontrolled proliferation, immortality, metastatic potential, rapid growth and proliferation rate, and certain morphological features. Often, cancer cells will be in the form of a tumor or mass, but such cells may exist alone within a subject, or may circulate in the blood stream as independent cells, such as leukemic or lymphoma cells. Examples of cancer as used herein include, but are not limited to, lung cancer, pancreatic cancer, bone cancer, skin cancer, head or neck cancer, cutaneous or intraocular melanoma, breast cancer, uterine cancer, ovarian cancer, peritoneal cancer, colon cancer, rectal cancer, colorectal adenocarcinoma, cancer of the anal region, stomach cancer, gastric cancer, gastrointestinal cancer, gastric adenocarcinoma, adrenocorticoid carcinoma, uterine cancer, carcinoma of the fallopian tubes, carcinoma of the endometrium, carcinoma of the vagina, carcinoma of the vulva, Hodgkin's Disease, esophageal cancer, gastroesophageal junction cancer, gastroesophageal adenocarcinoma, chondrosarcoma, cancer of the small intestine, cancer of the endocrine system, cancer of the thyroid gland, cancer of the parathyroid gland, cancer of the adrenal gland, sarcoma of soft tissue, Ewing's sarcoma, cancer of the urethra, cancer of the penis, prostate cancer, bladder cancer, testicular cancer, cancer of the ureter, carcinoma of the renal pelvis, mesothelioma, hepatocellular cancer, biliary cancer, kidney cancer, renal cell carcinoma, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. Some of the exemplified cancers are included in general terms and are included in this term. For example, urological cancer, a general term, includes bladder cancer, prostate cancer, kidney cancer, testicular cancer, and the like; and hepatobiliary cancer, another general term, includes liver cancers (itself a general term that includes hepatocellular carcinoma or cholangiocarcinoma), gallbladder cancer, biliary cancer, or pancreatic cancer. Both urological cancer and hepatobiliary cancer are contemplated by the present disclosure and included in the term “cancer.”

Also included within the term “cancer” is “solid tumor.” As used herein, the term “solid tumor” refers to those conditions, such as cancer, that form an abnormal tumor mass, such as sarcomas, carcinomas, and lymphomas. Examples of solid tumors include, but are not limited to, non-small cell lung cancer (NSCLC), neuroendocrine tumors, thyomas, fibrous tumors, metastatic colorectal cancer (mCRC), and the like. In some embodiments, the solid tumor disease is an adenocarcinoma, squamous cell carcinoma, large cell carcinoma, and the like.

In some embodiments, the cancer is esophageal cancer, gastroesophageal junction cancer, gastroesophageal adenocarcinoma, gastric cancer, chondrosarcoma, colorectal adenocarcinoma, breast cancer, ovarian cancer, head and neck cancer, elanoma, gastric adenocarcinoma, lung cancer, pancreatic cancer, renal cell carcinoma, hepatocellular carcinoma, cervical cancer, brain tumor, multiple myeloma, leukemia, lymphoma, prostate cancer, cholangiocarcinoma, endometrial cancer, small bowel adenocarcinoma, uterine sarcoma, or adrenocorticoid carcinoma. In some embodiments, the cancer is esophageal cancer, gastroesophageal junction cancer, gastroesophageal adenocarcinoma, colorectal adenocarcinoma, breast cancer, ovarian cancer, head and neck cancer, melanoma, gastric adenocarcinoma, lung cancer, pancreatic cancer, renal cell carcinoma, hepatocellular carcinoma, cervical cancer, brain tumor, multiple myeloma,leukemia, lymphoma, prostate cancer, cholangiocarcinoma, endometrial cancer, small bowel adenocarcinoma, uterine sarcoma, or adrenocorticoid carcinoma. In some embodiments, the cancer is breast cancer. In some embodiments, the cancer is colorectal adenocarcinoma. In some embodiments, the cancer is small bowel adenocarcinoma. In some embodiments, the cancer is hepatocellular carcinoma. In some embodiments, the cancer is head and neck cancer. In some embodiments, the cancer is renal cell carcinoma. In some embodiments, the cancer is ovarian cancer. In some embodiments, the cancer is prostate cancer. In some embodiments, the cancer is lung cancer. In some embodiments, the cancer is uterine sarcoma. In some embodiments, the cancer is esophageal cancer. In some embodiments, the cancer is endometrial cancer. In some embodiments, the cancer is cholangiocarcinoma. In some embodiments, each of the cancers is unresectable, advanced, refractory, recurrent, or metastatic.

As used herein, the terms “at least one compound of formula A” and “Compound A” each means a compound chosen from compounds having formula A:

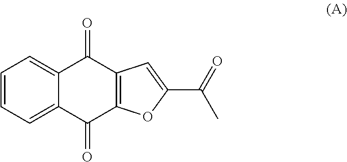 [SMILES: CC(=O)c1cc2c(o1)C(=O)c1ccccc1C2=O]

prodrugs, derivatives, pharmaceutically acceptable salts of any of the foregoing, and solvates of any of the foregoing.

In some embodiments, prodrugs and derivatives of compounds having formula A are STAT3 inhibitors. Non-limiting examples of prodrugs of compounds having formula A are, for example, the phosphoric ester and phosphoric diester described in U.S. pre-grant Publication No. 2012/0252763 as compound numbers 4011 and 4012 and also suitable compounds described in in U.S. Pat. No. 9,150,530. Non-limiting examples of derivatives of compounds having formula A include, for example, the derivatives disclosed in U.S. Pat. No. 8,977,803. The disclosures of U.S. pre-grant Publication No. 2012/0252763 and U.S. Pat. Nos. 9,150,530 and 8,977,803 are incorporated herein by reference in their entireties.

Compounds having formula A, shown below,

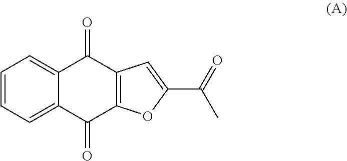 [SMILES: CC(=O)c1cc2c(o1)C(=O)c1ccccc1C2=O]

may also be known as 2-acetylnaphtho[2,3-b]furan-4,9-dione, napabucasin, or BBI608 and include tautorners thereof.

Suitable methods of preparing 2-acetylnaphtho[2,3-b]furan-4,9-dione, including its crystalline forms and additional cancer stemness inhibitors, are described in the co-owned PCT applications published as WO 2009/036099, WO 2009/036101, WO 2011/116398, WO 2011/116399, and WO 2014/169078; the contents of each of these applications are incorporated herein by reference in their entireties.

As used herein, the terms “at least one compound of formula B” and “Compound B” each means a compound chosen from compounds having formula (B):

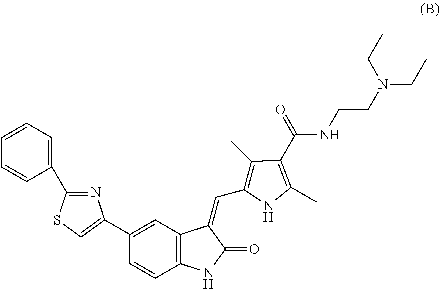 [SMILES: CCN(CC)CCNC(=O)c1c(C)[nH]c(/C=C2\C(=O)Nc3ccc(-c4csc(-c5ccccc5)n4)cc32)c1C]

prodrugs, derivatives, pharmaceutically acceptable salts of any of the foregoing, and solvates of any of the foregoing.

In some embodiments, compounds having formula B and derivatives thereof are kinase-targeting agents or kinase inhibitors. In some embodiments, compounds having formula B and derivatives thereof are cancer stem cell pathway kinase (CSCPK) inhibitors. In some embodiments, compounds having formula B and derivatives thereof are inhibitors of STK33, MELK, AXL, p70S6K, and PDGFRα. In some embodiments, at least one compound chosen from compounds having formula B and derivatives thereof is a STK33 inhibitor. In some embodiments, at least one compound chosen from compounds having formula B and derivatives thereof is a MELK inhibitor. In some embodiments, at least one compound chosen from compounds having formula B and derivatives thereof is an AXL inhibitor. In some embodiments, at least one compound chosen from compounds having formula B and derivatives thereof is a p70S6K inhibitor. In some embodiments, at least one compound chosen from compounds having formula B and derivatives thereof is a PDGFRα inhibitor. In some embodiments, at least one compound chosen from compounds having formula B and derivatives thereof inhibits NANOG expression. Non-limiting examples of compounds having formula B and derivatives thereof include, for example, the derivatives disclosed in U.S. Pat. No. 8,299,106 and PCT Patent Application Publication No. WO2014160401. The disclosures of U.S. Pat. No. 8,299,106 and PCT Patent Application Publication No. WO2014160401 are incorporated herein by reference in their entireties. In some embodiments, the kinase targeting agent or kinase inhibitor or CSCPK inhibitor is chosen from

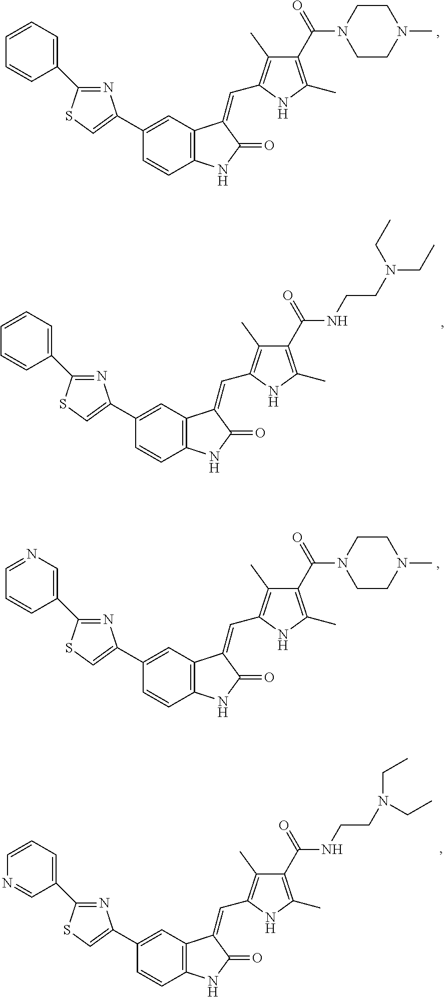 [SMILES: Cc1[nH]c(/C=C2\C(=O)Nc3ccc(-c4csc(-c5ccccc5)n4)cc32)c(C)c1C(=O)N1CCN(C)CC1 ; CCN(CC)CCNC(=O)c1c(C)[nH]c(/C=C2\C(=O)Nc3ccc(-c4csc(-c5ccccc5)n4)cc32)c1C ; Cc1[nH]c(/C=C2\C(=O)Nc3ccc(-c4csc(-c5cccnc5)n4)cc32)c(C)c1C(=O)N1CCN(C)CC1 ; CCN(CC)CCNC(=O)c1c(C)[nH]c(/C=C2\C(=O)Nc3ccc(-c4csc(-c5cccnc5)n4)cc32)c1C]

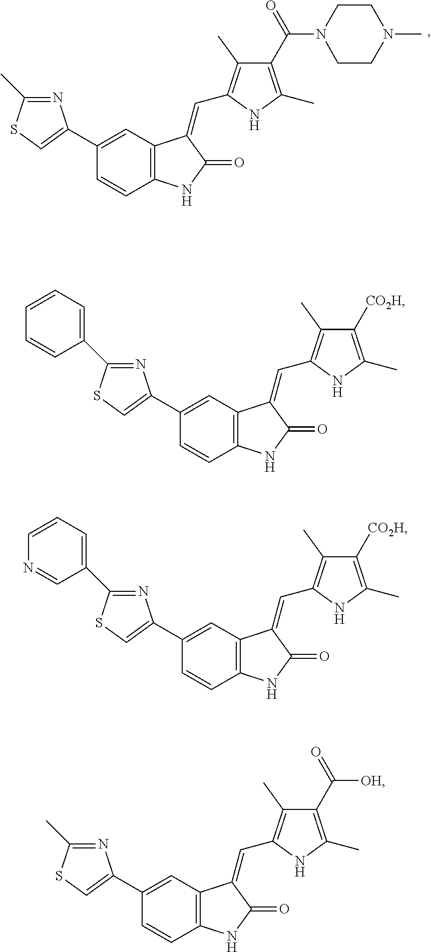

prodrugs of any of the foregoing, derivatives of any of the foregoing, pharmaceutically acceptable salts of any of the foregoing, and solvates of any of the foregoing.

Suitable methods of preparing compounds having formula B and derivatives thereof are described in U.S. Pat. No. 8,299,106 and PCT Patent Application Publication No. WO2014160401; the contents of each application are incorporated herein by reference in their entireties.

The term “salt(s),” used herein, includes acidic and/or basic salts formed with inorganic and/or organic acids and bases. As used herein, the term “pharmaceutically acceptable salt” refers to those salts which are, within the scope of sound medical judgment, suitable for use in contact with the tissues of subjects without undue toxicity, irritation, allergic response and/or the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19.

Pharmaceutically acceptable salts may be formed with inorganic or organic acids. Non-limiting examples of suitable inorganic acids include hydrochloric acid, hydrobromic acid, phosphoric acid, sulfuric acid, and perchloric acid. Non-limiting examples of suitable organic acids include acetic acid, oxalic acid, maleic acid, tartaric acid, citric acid, succinic acid, and malonic acid. Other non-limiting examples of suitable pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydrolodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, and salicylic acid.

Salts may be prepared in situ during the isolation and purification of the disclosed compound, or separately, such as by reacting the compound with a suitable base or acid, respectively. Non-limiting examples of pharmaceutically acceptable salts derived from bases include alkali metal, alkaline earth metal, ammonium and N⁺(C₁₋₄alkyl)₄ salts. Non -limiting examples of suitable alkali or alkaline earth metal salts include sodium, lithium, potassium, calcium, magnesium, iron, zinc, copper, manganese, and aluminum salts. Further non-limiting examples of suitable pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate, examples of suitable organic bases from which salts may be derived include primary amines, secondary amines, tertiary amines, substituted amines including naturally occurring substituted amines, cyclic amines, and basic ion exchange resins, such as isopropylamine, trimethylamine, diethylamine, triethylamine, tripropylamine, and ethanolamine. In some embodiments, pharmaceutically acceptable base addition salts can be chosen from ammonium, potassium, sodium, calcium, and magnesium salts.

The term “solvate” represents an aggregate that comprises one or more molecules of a compound of the present disclosure with one or more molecules of a solvent or solvents. Solvates of the compounds of the present disclosure include, for example, hydrates.

The at least one compound disclosed herein may be in the form of a pharmaceutical composition. In some embodiments, the pharmaceutical compositions may comprise the at least one compound of formula A and at least one pharmaceutically acceptable carrier. In some embodiments, the pharmaceutical compositions may comprise the at least one compound of formula B and at least one pharmaceutically acceptable carrier. In some embodiments, the pharmaceutical compositions may comprise one or more compounds and at least one pharmaceutically acceptable carrier, where the one or more compounds are capable of being converted into the at least one compound of formula A in a subject (i.e., a prodrug). In some embodiments, the pharmaceutical compositions may comprise one or more compounds and at least one pharmaceutically acceptable carrier, where the one or more compounds are capable of being converted into the at least one compound of formula B in a subject (i.e., a prodrug).

The term “carrier” as used herein means a pharmaceutically acceptable material, composition or vehicle, such as, for example, a liquid or solid filler, diluent, excipient, solvent or encapsulating material involved in or capable of carrying or transporting the subject pharmaceutical compound from one organ, or portion of the body, to another organ, or portion of the body. Each carrier must be “acceptable” in the sense of being compatible with the other ingredients of the formulation and not injurious to the patient. Non-limiting examples of pharmaceutically acceptable carriers, carriers, and/or diluents include: sugars, such as lactose, glucose and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers, and lubricants, such as sodium lauryl sulfate, magnesium stearate, and polyethylene oxide-polypropylene oxide copolymer as well as coloring agents, release agents, coating agents, sweetening, flavoring and perfuming agents, preservatives, and antioxidants can also be present in the compositions.

In some embodiments, the at least one compound of formula A may be administered in an amount ranging from about 80 mg to about 1500 mg. In some embodiments, the at least one compound may be administered in an amount ranging from about 160 mg to about 1000 mg. In some embodiments, the at least one compound of formula A may be administered in an amount ranging from about 300 mg to about 700 mg a day. In some embodiments, the at least one compound of formula A may be administered in an amount ranging from about 700 mg to about 1200 mg. In some embodiments, the at least one compound of formula A may be administered in an amount ranging from about 800 mg to about 1100 mg. In some embodiments, the at least one compound of formula A may be administered in an amount ranging from about 850 mg to about 1050 mg. In some embodiments, the at least one compound of formula A may be administered in an amount ranging from about 960 mg to about 1000 mg. In some embodiments, the total amount of the at least one compound of formula A is administered once daily. In some embodiments, the at least one compound of formula A is administered in a dose of about 480 mg daily. In some embodiments, the at least one compound of formula A is administered in a dose of about 960 mg daily. In some embodiments, the at least one compound of formula A is administered in a dose of about 1000 mg daily. In some embodiments, the total amount of the at least one compound of formula A is administered in divided doses more than once daily, such as twice daily (BID) or more often. In some embodiments, the at least one compound of formula A may be administered in an amount ranging from about 80 mg twice daily to about 750 mg twice daily. In some embodiments, the at least one compound may be administered in an amount ranging from about 80 mg twice daily to about 500 mg twice daily. In some embodiments, the at least one compound of formula A is administered in a dose of about 240 mg twice daily. In some embodiments, the at least one compound of formula A is administered in a dose of about 480 mg twice daily. In some embodiments, the at least one compound of formula A is administered in a dose of about 500 mg twice daily. In some embodiments, the at least one compound of formula A is administered orally.

In some embodiments, the cancer stemness inhibitor may be administered in an amount ranging from about 300 mg to about 700 mg. In some embodiments, the cancer stemness inhibitor may be administered in an amount ranging from about 700 mg to about 1200 mg. In some embodiments, the cancer stemness inhibitor may be administered in an amount ranging from about 800 mg to about 1100 mg. In some embodiments, the cancer stemness inhibitor may be administered in an amount ranging from about 850 mg to about 1050 mg. In some embodiments, the cancer stemness inhibitor may be administered in an amount ranging from about 960 mg to about 1000 mg. In some embodiments, the total amount of the cancer stemness inhibitor is administered once daily. In some embodiments, the cancer stemness inhibitor is administered in a dose of about 480 mg daily. In some embodiments, the cancer stemness inhibitor is administered in a dose of about 960 mg daily. In some embodiments, the cancer stemness inhibitor is administered in a dose of about 1000 mg daily. In some embodiments, the total amount of the cancer stemness inhibitor is administered in divided doses more than once daily, such as twice daily (BID) or more often. In some embodiments, the cancer stemness inhibitor is administered in a dose of about 240 mg twice daily. In some embodiments, the cancer stemness inhibitor is administered in a dose of about 480 mg twice daily. In some embodiments, the cancer stemness inhibitor is administered in a dose of about 500 mg twice daily. In some embodiments, the cancer stemness inhibitor is administered orally.

In some embodiments, the at least one compound of formula B may be administered in an amount ranging from about 20 mg to about 600 mg. In some embodiments, the at least one compound of formula B may be administered in an amount ranging from about 50 mg to about 500 mg. In some embodiments, the at least one compound of formula B may be administered in an amount ranging from about 80 mg to about 400 mg. In some embodiments, the at least one compound of formula B may be administered in an amount ranging from about 80 mg to about 300 mg. In some embodiments, the at least one compound of formula B is administered once daily. In some embodiments, the at least one compound of formula B is administered in a dose of about 100 mg daily. In some embodiments, the at least one compound of formula B is administered in a dose of about 200 mg daily. In some embodiments, the at least one compound of formula B is administered in a dose of about 300 mg daily. In some embodiments, the total amount of the at least one compound of formula B is administered in a single daily dose. In some embodiments, the total amount of the at least one compound of formula B is administered in divided doses more than once daily, such as twice daily (BID) or more often. In some embodiments, the at least one compound of formula B is administered in a dose of about 100 mg once daily. In some embodiments, the at least one compound of formula B is administered in a dose of about 200 mg once daily. In some embodiments, the at least one compound of formula B is administered orally.

In some embodiments, the kinase-targeting agent or kinase inhibitor may be administered in an amount ranging from about 20 mg to about 600 mg. In some embodiments, the kinase-targeting agent or kinase inhibitor may be administered in an amount ranging from about 50 mg to about 500 mg. In some embodiments, the kinase-targeting agent or kinase inhibitor may be administered in an amount ranging from about 80 mg to about 400 mg. In some embodiments, the kinase-targeting agent or kinase inhibitor may be administered in an amount ranging from about 80 mg to about 300 mg. In some embodiments, the kinase-targeting agent or kinase inhibitor is administered once daily. In some embodiments, the kinase-targeting agent or kinase inhibitor is administered in a dose of about 100 mg daily. In some embodiments, the kinase targeting agent or kinase inhibitor is administered in a dose of about 200 mg daily. In some embodiments, the kinase-targeting agent or kinase inhibitor is administered in a dose of about 300 mg daily. In some embodiments, the total amount of the kinase targeting agent or kinase inhibitor is administered in a single daily dose. In some embodiments, the total amount of the kinase-targeting agent or kinase inhibitor is administered in divided doses more than once daily, such as twice daily (BID) or more often. In some embodiments, the kinase-targeting agent or kinase inhibitor is administered in a dose of about 100 mg once daily. In some embodiments, the kinase-targeting agent or kinase inhibitor is administered in a dose of about 200 mg once daily. In some embodiments, the kinase-targeting agent or kinase inhibitor is administered orally.

In some embodiments, said cancer stemness inhibitor s administered orally at a dose in a range from about 80 mg to about 960 mg twice daily, or at a dose in a range from about 160 mg to about 240 mg twice daily, or a dose of about 480 mg twice daily, and said kinase targeting agent is administered orally at a dose in a range from about 100 mg to about 600 mg once daily, or at a range of 200 mg once daily. In some embodiments, said cancer stemness inhibitor is administered orally at a dose of about 480 mg twice daily, and said kinase-targeting agent is administered orally at a dose of about 300 mg once daily.

Pharmaceutical compositions disclosed herein that are suitable for oral administration may be in the form of capsules, cachets, pills, tablets, lozenges (using a flavored basis, usually sucrose and acacia or tragacanth), powders, granules, a solution in an aqueous or non-aqueous liquid, a suspension in an aqueous or non-aqueous liquid, an oil-in-water emulsion, a water-in-oil emulsion, an elixir, a syrup, pastilles (using an inert base, such as gelatin, glycerin, sucrose, and/or acacia) and/or mouthwashes, each containing a predetermined amount of the at least one compound of the present disclosure.

A pharmaceutical composition disclosed herein may be administered as a bolus, electuary, or paste.

Solid dosage forms for oral administration (capsules, tablets, pills, dragees, powders, granules and the like) may be mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose, and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polypropylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type also may be employed as fillers in soft and hard-filled gelatin capsules using such excipients as lactose or milk sugars, as well as high molecular weight polyethylene glycols and the like.

Liquid dosage forms for oral administration may include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, and fatty acid esters of sorbitan, and mixtures thereof. Additionally, cyclodextrins, e.g., hydroxypropyl-β-cyclodextrin, may be used to solubilize compounds.

The pharmaceutical compositions also may include adjuvants such as wetting agents, emulsifying and suspending agents, sweetening, flavoring, coloring, perfuming, and preservative agents. Suspensions, in addition to the compounds according to the disclosure, may contain suspending agents as, such as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Pharmaceutical compositions disclosed herein, for rectal or vaginal administration may be presented as a suppository, which may be prepared by mixing one or more compounds according to the present disclosure with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the compounds of the present disclosure. Pharmaceutical compositions which are suitable for vaginal administration also may include pessaries, tampons, creams, gels, pastes, foams, or spray formulations containing carriers that are known in the art to be appropriate.

Dosage forms for the topical or transdermal administration of a pharmaceutical composition or pharmaceutical tablet of the present disclosure may include powders, sprays, ointments, pastes, creams, lotions, gels, solutions, patches, and inhalants. The pharmaceutical composition or pharmaceutical tablet may be mixed under sterile conditions with a pharmaceutically acceptable carrier, and with any preservatives, buffers, or propellants which may be required.

The ointments, pastes, creams and gels may contain, in addition to the pharmaceutical composition or pharmaceutical tablet of the present disclosure, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays may contain, in addition to a pharmaceutical composition or a pharmaceutical tablet of the present disclosure, excipients such as lactose, talc, silicic acid, aluminum hydroxide, calcium silicates, and polyamide powder, or mixtures of these substances. Additionally, sprays may contain customary propellants, such as chlorofluorohydrocarbons and volatile unsubstituted hydrocarbons, such as butane and propane.

Ophthalmic formulations, eye ointments, powders, solutions and the like, are also contemplated as being within the scope of the present disclosure.

Compositions suitable for parenteral administration may comprise at least one more pharmaceutically acceptable sterile isotonic aqueous or nonaqueous solutions, dispersions, suspensions, emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In various embodiments, a composition described herein includes at least one compound chosen from compounds of formula A and pharmaceutically acceptable salts and solvates thereof and one or more surfactants. In some embodiments, the surfactant is sodium lauryl sulfate (SLS), sodium dodecyl sulfate (SDS), or one or more polyoxylglycerides. For example, the polyoxylglyceride can be lauroyl polyoxylglycerides (sometimes referred to as Gelucire™) or linoleoyl polyoxylglycerides (sometimes referred to as Labrafil™). Examples of such compositions are shown in PCT Patent Application No. PCT/US2014/033566, the contents of which are incorporated herein in their entireties.

The present invention provides further embodiments of suitable pharmaceutical formulations having selected particle size distribution and methods for identifying an optimum particle size distribution, suitable drug regimen, dosage and interval, suitable methods of preparing 2-acetylnaphtho[2,3-b]furan-4,9-dione including their crystalline forms, and further specific suitable cancer stemness inhibitors and kinase inhibitors as described in the co-owned PCT applications published as WO 2009/036099, WO 2009/036101, WO 2011/116398, WO 2011/116399, WO 2014/169078, and WO 2009/033033, the contents of which are incorporated by reference herein in their entirety.

In various embodiments, a composition described herein includes at least one compound chosen from compounds of formula B and pharmaceutically acceptable salts and solvates thereof and one or more surfactants. In some embodiments, the surfactant is sodium lauryl sulfate (SLS), sodium dodecyl sulfate (SOS), or one or more polyoxyiglycerides. For example, the polyoxylglyceride can be lauroyl polyoxylglycerides (sometimes referred to as Gelucire™) or linoleoyl polyoxylgiycerides (sometimes referred to as Labrafil™).

In some embodiments, the compounds or pharmaceutical compositions described herein are administered in combination with any of a variety of known therapeutics, including for example, chemotherapeutic and other anti-neoplastic agents, anti-inflammatory compounds, and/or immunosuppressive compounds. In some embodiments, the compounds, products, and/or pharmaceutical compositions described herein are useful in conjunction with any of a variety of known treatments including, by way of non-limiting example, surgical treatments and methods, radiation therapy, chemotherapy, and/or hormone or other endocrine-related treatment.

Disclosed herein are methods of inhibiting, reducing, and/or diminishing CSC survival and/or self-renewal comprising administering a therapeutically effective amount of at least one pharmaceutical composition comprising at least one compound of formula A in combination with a therapeutically effective amount of at least one pharmaceutical composition comprising at least one compound of formula B. Also disclosed herein are methods of inhibiting, reducing, and/or diminishing CSC survival and/or self-renewal comprising administering a therapeutically effective amount of at least one compound of formula A in combination with a therapeutically effective amount of at least one compound of formula B.

Also disclosed herein are methods of treating at least one cancer that is refractory to conventional chemotherapies and/or targeted therapies in a subject comprising administering a therapeutically effective amount of at least one compound of formula A in combination with a therapeutically effective amount of at least one compound of formula B. In some embodiments, the at least one compound of formula A is included in a pharmaceutical composition. In some embodiments, the at least one compound of formula B is included in a pharmaceutical composition.

Disclosed herein are methods of treating recurrent cancer in a subject that has failed surgery, oncology therapy (e.g., chemotherapy), and/or radiation therapy, comprising administering a therapeutically effective amount of at least one compound of formula A in combination with a therapeutically effective amount of at least one compound of formula B. In some embodiments, the at least one compound of formula A is included in a pharmaceutical composition. In some embodiments, the at least one compound of formula B is included in a pharmaceutical composition.

Also disclosed herein are methods of treating or preventing cancer metastasis in a subject, comprising administering a therapeutically effective amount of at least one compound of formula A in combination with a therapeutically effective amount of at least one compound of formula B. In some embodiments, the at least one compound of formula A is included in a pharmaceutical composition. In some embodiments, the at least one compound of formula B is included in a pharmaceutical composition.

Also disclosed herein are methods of preventing relapse or suppressing regrowth or recurrent of cancer in a subject, comprising administering a therapeutically effective amount of at least one compound of formula A in combination with a therapeutically effective amount of at least one compound of formula B. In some embodiments the method is a part of an adjuvant therapy. In some embodiments, the method comprises administering a treatment combination of the present disclosure after or currently with a primary treatment of cancer. In some embodiments, the primary treatment is chosen from chemotherapies, radiation therapies, hormone therapies, targeted therapies, or biological therapies. In some embodiments, the at least one compound of formula A is included in a pharmaceutical composition. In some embodiments, the at least one compound of formula B is included in a pharmaceutical composition.

Disclosed herein are methods of treating cancer in a subject comprising administering a therapeutically effective amount of at least one compound of formula A in combination with a therapeutically effective amount of at least one compound of formula B. In some embodiments, the at least one compound of formula A is included in a pharmaceutical composition. In some embodiments, the at least one compound of formula B is included in a pharmaceutical composition.

In some embodiments, the cancer is esophageal cancer, gastroesophageal junction cancer, gastroesophageal adenocarcinoma, gastric cancer, chondrosarcoma, colorectal adenocarcinoma, breast cancer, ovarian cancer, head and neck cancer, melanoma, gastric adenocarcinoma, lung cancer, pancreatic cancer, renal cell carcinoma, hepatocellular carcinoma, cervical cancer, brain tumor, multiple myeloma, leukemia, lymphoma, prostate cancer, cholangiocarcinoma, endometrial cancer, small bowel adenocarcinoma, uterine sarcoma, or adrenocorticoid carcinoma. In some embodiments, the cancer is esophageal cancer, gastroesophageal junction cancer, gastroesophageal adenocarcinoma, colorectal adenocarcinoma, breast cancer, ovarian cancer, head and neck cancer, melanoma, gastric adenocarcinoma, lung cancer, pancreatic cancer, renal cell carcinoma, hepatocellular carcinoma, cervical cancer, brain tumor, multiple myeloma, leukemia, lymphoma, prostate cancer, cholangiocarcinoma, endometrial cancer, small bowel adenocarcinoma, uterine sarcoma, or adrenocorticoid carcinoma. In some embodiments, the cancer is breast cancer. In some embodiments, the cancer is colorectal adenocarcinoma. In some embodiments, the cancer is small bowel adenocarcinoma. In some embodiments, the cancer is hepatocellular carcinoma. In some embodiments, the cancer is head and neck cancer. In some embodiments, the cancer is renal cell carcinoma. In some embodiments, the cancer is ovarian cancer. In some embodiments, the cancer is prostate cancer. In some embodiments, the cancer is lung cancer. In some embodiments, the cancer is uterine sarcoma. In some embodiments, the cancer is esophageal cancer. In some embodiments, the cancer is endometrial cancer. In some embodiments, the cancer is cholangiocarcinoma.

In some embodiments, the cancer may be unresectable. In some embodiments, the cancer may be advanced. In some embodiments, the cancer may be refractory. In some embodiments, the cancer may be recurrent. In some embodiments, the cancer may be metastatic. In some embodiments, the cancer may be associated with overexpression of STAT3. In some embodiments, the cancer may be associated with nuclear β-catenin localization.

### EXAMPLES

Examples are provided below to further illustrate different features of the present invention. The examples also illustrate useful methodology for practicing the invention. These examples do not limit the claimed invention.

The methods disclosed herein comprise administering to a subject in need thereof a therapeutically effective amount of at least one compound of formula A in combination with a therapeutically effective amount of at least one compound of formula B.

### Example 1

### Enhanced Inhibition of Phospho-STAT3 Following In Vitro Combination Treatment with Compound A and Compound B

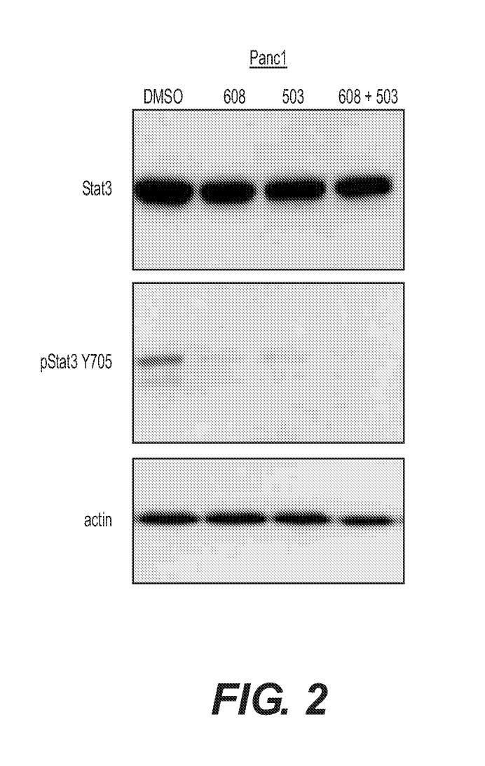

The effects of Compound A, Compound B, and a combination thereof to inhibit phospho-STAT3 in cancer cells were studied. For these studies, Panc-1 pancreatic cancer cells were treated with Compound A alone, with Compound B alone, or with Compound A and Compound B in combination. Referring to FIG. 2, for the combination treatment, human pancreatic (Panc-1) cancer cells were incubated with Compound B (5 μM) for 20 hours then co-treated with Compound B (5 μM) and Compound A (1 μM) for 4 hours. Cell lysates were then prepared and examined for levels of STAT3, p-STAT3, and β-actin by Western blotting.

As shown in FIG. 2, the treatment with Compound A and Compound B in combination resulted in enhanced inhibition of p-STAT3 in comparison to treatments with Compound A alone, or with Compound B alone.

### Example 2

### Enhanced Inhibition of Nanog Following In Vitro Treatment Combination with Compound A and Compound B

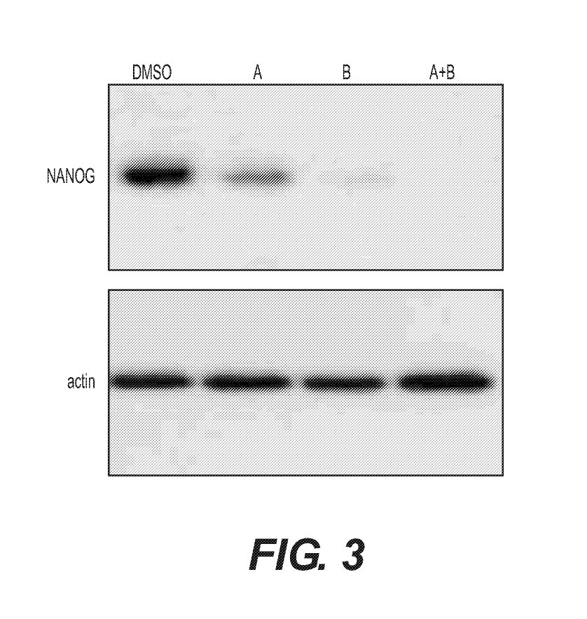

The effect of treatment combination with Compound A and Compound B to inhibit the stemness-associated transcription factor Nanog in cancer cells were studied. For these studies, MKN28 gastric cancer cells were treated with Compound A alone, or with Compound B alone, or with Compound A and Compound B in combination. Referring to FIG. 3, human gastric (MKN28) cancer cells were co-treated with Compound B (5 μM), Compound A (1 μM), or Compound B and Compound A (5 μM and 1 μM, respectively) for 24 hours. Cell lysates were then prepared and examined for levels of Nanog and β-actin by Western blotting.

As shown in FIG. 3, treatment with Compound A and Compound B in combination resulted in enhanced inhibition of Nanog protein expression when compared to the treatment with Compound A alone, or with Compound B alone.

### Example 3

### Treatment Combination with Compound A and Compound B In Vitro Enhanced the Inhibition of Bulk Cancer Cell Colony Formation

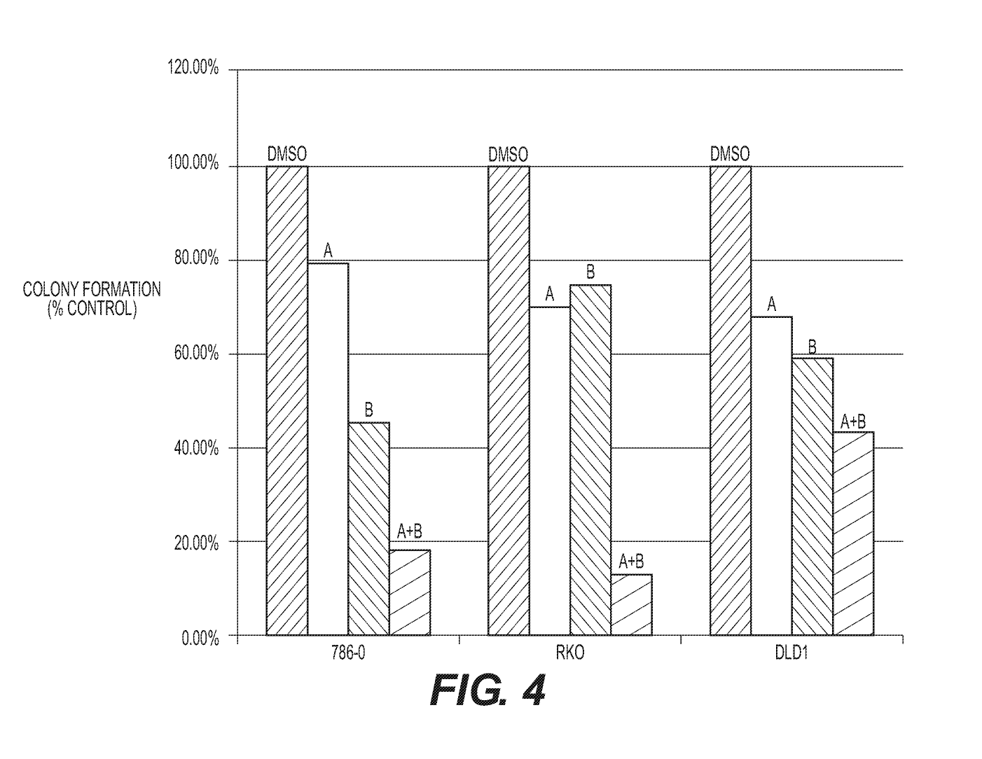

The treatment combination with Compound A and Compound B on the ability of bulk cancer cells to undergo clonogenic expansion was examined by colony formation assay. For these studies, human kidney cancer cells (786-0), human colon cancer cells (RKO), and human colon cancer cells (DLD-1) were treated with Compound A alone, with Compound B alone, or with Compound A and Compound B in combination. Referring to FIG. 4, 786-0 human kidney cancer cells, WO human colon cancer cells, and DLD-1 human colon cancer cells were seeded onto 6-well plates at 1000 cells/well, 24 hours after plating, cells were exposed to vehicle, Compound A (for 4 hours), Compound B (for 24 hours), or Compound A and Compound B (for 24 hours) at the indicated doses. Cells were then cultured for 10-14 days, fixed, and stained with Giemsa.

As shown in FIG. 4, the treatment with Compound A and Compound B in combination resulted in enhanced inhibition of 786-0, RKO and DLD-1 colony formation in comparison to treatment with Compound A alone or with Compound B alone. Similar data were also observed with SW480 colon cancer cells, AGS gastric cancer cells and MKN28 gastric cancer cells.

### Example 4

### Enhanced Inhibition of Cancer Stem Cell Sphere Formation Following Treatment Combination with Compound A and Compound B In Vitro

The effect of combination treatment with Compound A and Compound B on cancer stem cell sphere formation (i.e. spherogenesis) was studied. For these studies, DLD-1, RKO colon cancer cells, and ACHN kidney cancer cells were dissociated with Accutase® cell detachment solution, washed with PBS, and resuspended in CSC media at a concentration of 1×10³ cells/mL. After being cultured for 72 hours, the resulting CSCs were incubated with Compound A (0.5-1.0 μM), Compound B (1.25-2.5 μM), or both Compound A and Compound B (0.5-1.0 μM and 1.25-2.5 μM, respectively). CSC spheres were then allowed to grow for 72 hours, after which the cell viability was determined using a CellTiter-Glo® luminescent cell viability assay (Promega).

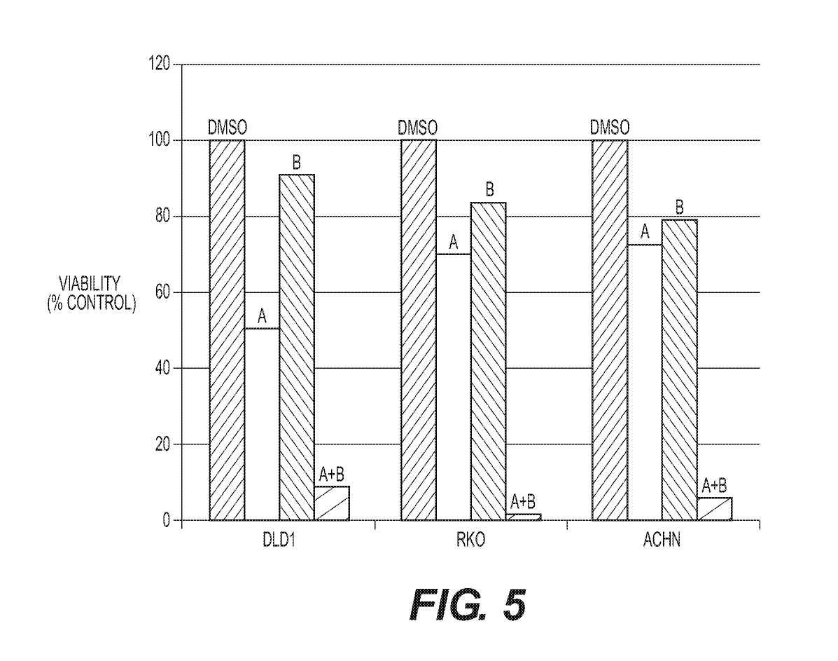

As shown in FIG. 5, the treatment combination of Compound A and Compound B resulted in an enhanced inhibition of DLD-1, RKO, and ACHN CSC viability as compared to treatment with Compound A alone or with Compound B alone.

In summary, the studies described in Examples 1-4 demonstrated that Compound A and Compound B act synergistically in vitro, and these data suggest significant potential for combined therapy using Compound A and Compound B for a wide variety of human cancers.

### Example 5

### Enhanced Knockdown of Pharmacodynamic Markers of Compound A and Compound B Treatment Following In Vivo Combination Drug Therapy

The effect of a treatment combination of Compound A and Compound B on the levels of various pharmacodynamic (PD) markers for the agents in vivo was studied. A mouse xenograft model of human colon cancer (SW480) was used.

Levels of STK33 and Nanog were examined using immunofluorescence staining of the xenograft tissue. Female nude mice were inoculated subcutaneously with 8×10⁶ human SW480 colon cancer cells. Animals were treated orally daily with vehicle, Compound A (100 mg/kg), Compound B (50 mg/kg), or Compound A and Compound B (100 mg/kg and 50 mg/kg, respectively) for a total of 14 doses. At the end of treatment, the tumors were harvested from euthanized mice. Part of the dissected tumors were fixed overnight in 3.7% neutral buffered formaldehyde at 4° C., and then paraffin embedded, cut to four micron sections, and affixed onto positively charged slides. After being baked and deparaffinized, the slides with tumor or control tissues were incubated in 10mM pH6.0 sodium citrate solution for antigen retrieval at 98° C. Afterwards, the slides were probed with the primary antibodies against P-STAT3 (Tyr705) (rabbit, Cell Signaling, 1:100), STK33 (mouse, Abnova, 1:200), Nanog (rabbit, Santa Cruz, 1:100) at 4° C. overnight, and then AlexaFluor fluorescent dye-conjugated secondary antibodies (Invitrogen, 1:300) at room temperature for one hour. After being mounted with ProLong mounting medium containing DAPI (Invitrogen), the slides were examined on a Zeiss Axis Imager M2 upright fluorescence microscope with a 20× objective and analyzed with Zen software.

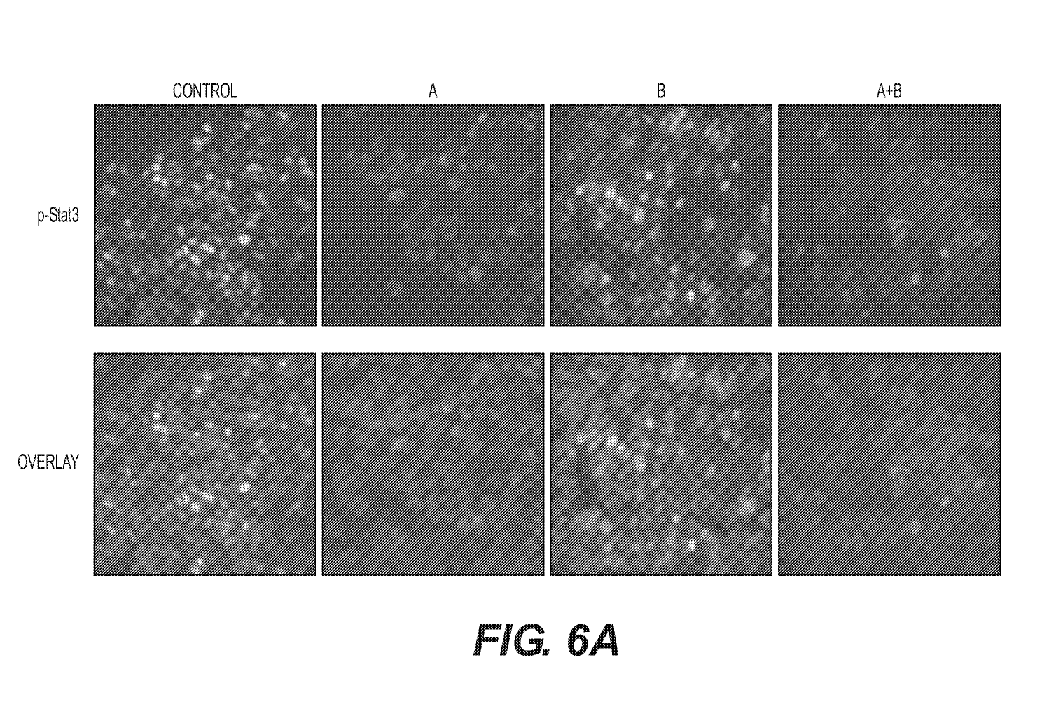

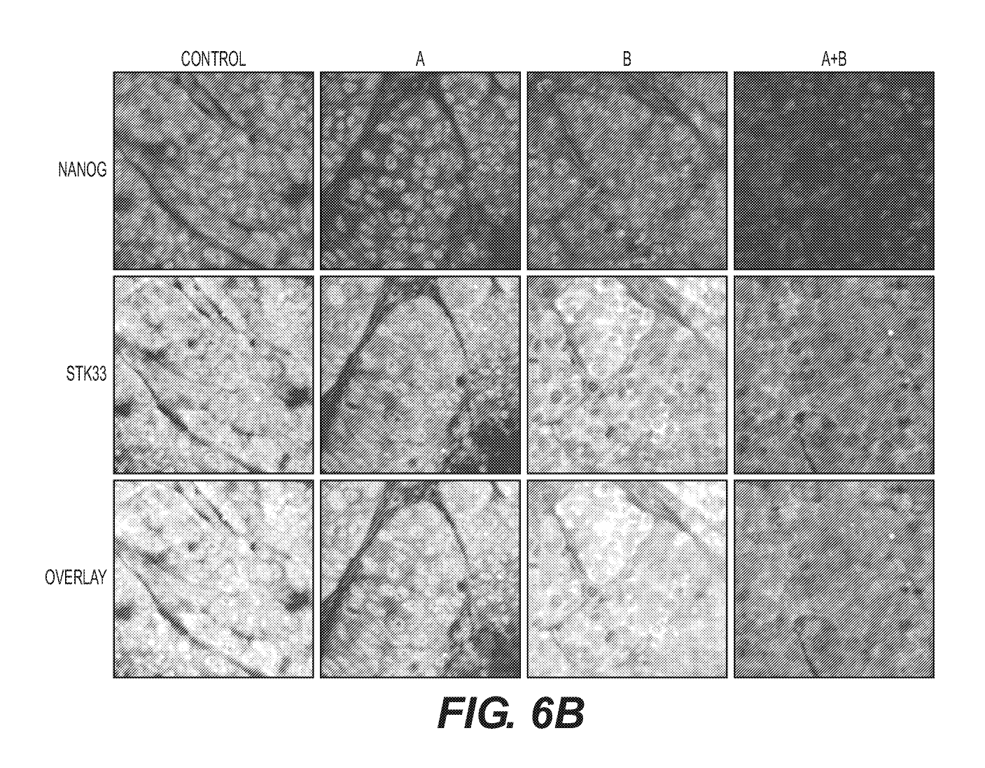

As shown in FIG. 6A, treatment with Compound A, but not Compound B, significantly reduced cellular levels of p-STAT3. As shown in FIG. 6B, treatment with Compound A also reduced cytoplasmic levels of both STK33 and Nanog (FIG. 6B). By contrast, treatment with Compound B reduced nuclear levels of both proteins. The treatment combination of Compound A and Compound B was associated with an enhanced reduction in both cytoplasmic and nuclear levels of STK33 and Nanog in the xenograft tissue.

As shown in FIGS. 6(A)-(B), PD markers for Compound A and/or Compound B in xenograft tissue were analysed by immunofluorescence staining (FIG. 6(A) and FIG. 6(B)). The treatment combination of Compound A and Compound B resulted in enhanced inhibition of p-STAT3, STK33 and Nanog levels in human SW480 colon cancer xenograft tissue as compared to treatment with Compound A alone or with Compound B alone.

### Example 6

### Enhanced In Vivo Anti-Cancer Stem Activity Following Combined Treatment with Compound A and Compound B

The ability of the Compound A+Compound B combination to target CSCs in vivo was examined using the mouse xenograft model of human colon cancer (SW480) described above in Example 5. Specifically, mice were treated orally daily with vehicle, Compound A (100 mg/kg), Compound B (50 mg/kg), or Compound A+Compound B (100 mg/kg and 50 mg/kg, respectively) for a total of 14 doses. After 14 days of treatment, the tumors were harvested from the euthanized mice. Portions of the tumor tissues were dissociated into single cell suspensions by the enzymatic digestion with DMEM (Gibco) containing 200 U/mL Collagenase (Sigma) and 100 U/mL DNAse I (Sigma) at 37° C. for 30 minutes. The resulting cells were then filtered through 40 μm strainers and incubated 5 min at room temperature in ACK lysis buffer (Thermo Fisher) to remove the red blood cells, 1000 live tumor cells, as assessed by Trypan blue (Gibco) staining, were suspended in 1 mL sphere medium and plated on low-attachment cell culture 12-well plate in triplicate. The cancer sphere culture medium comprised B-27 (Gibco), 20 ng/ml EGF (R&D), 10 ng/ml basicFGF (bFGF, R&D), 0.4% BSA Gemini, and 0.3% agarose in DMEM/F12 (Gibco). The resulting tumor spheres were counted after 10 days. Spheres with >50 cells were scored.

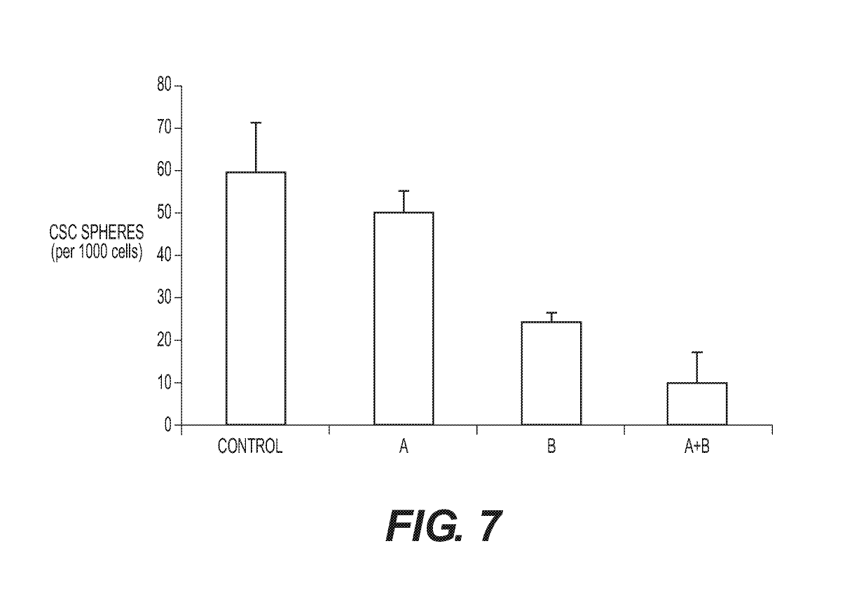

As shown in FIG. 7, treatment of mice harboring xenograft tumors with the Compound A and Compound B combination enhanced the reduction in the number of CSCs as compared to animals treated with Compound A or Compound B alone.

### Example 7

### Combined Treatment with Compound A and Compound B Results in Enhanced Anti-Tumor Activity in Multiple Mouse Xenograft Models of Human Cancer

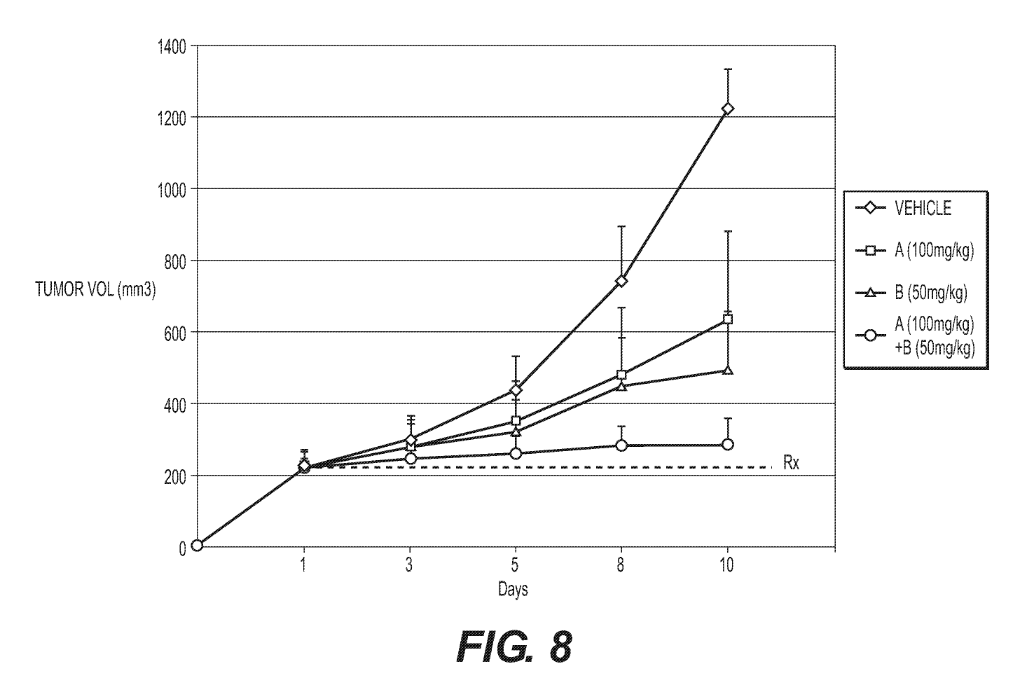

In the mouse xenograft model of human colon cancer, SW480 cells were inoculated subcutaneously into male athymic nude mice (8×10⁶ cells/mouse) and allowed to form palpable tumors. Once the tumors reached approximately 200 mm³, the animals were treated orally with vehicle, Compound A (100 mg/kg), Compound B (50 mg/kg), or Compound A and Compound B (100 mg/kg and 50 mg/kg, respectively) as indicated in FIG. 8. The animals received a total of 9 doses. Treatment with Compound A and Compound B as a combination therapy synergistically inhibited tumor growth as compared to animals treated with Compound A or Compound B alone. Tumor growth inhibition for the Compound A+Compound B combination was calculated to be 77% (p=0.0005). These data suggest that Compound A and Compound B can be safely dosed in a regimen that is effective, supporting further clinical evaluation for the treatment of human colon cancer.

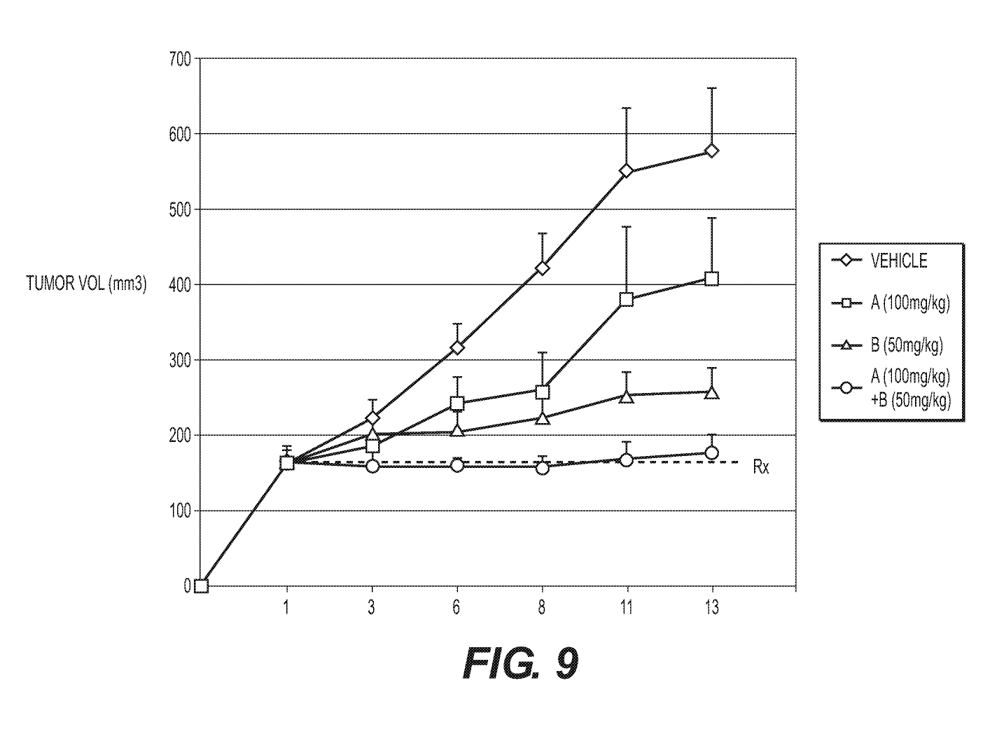

In the mouse xenograft model of human gastric cancer, MKN-45 cells were inoculated subcutaneously into male athymic nude mice (8×10⁶ cells/mouse) and allowed to form palpable tumors. Once the tumors reached approximately 170 mm³, the animals were treated orally with vehicle, Compound A (100 mg/kg), Compound B (50 mg/kg), or Compound A and Compound B (100 mg/kg and 50 mg/kg), respectively, as indicated in FIG. 9. All regimens were administered daily for a total of 12 doses. Tumor size was evaluated periodically during treatment. Each point represents the mean+SEM of 5 tumors.

Treatment with Compound A and Compound B as a combination therapy enhanced the inhibition of tumor growth in comparison to animals treated with Compound A or Compound B alone. Tumor growth inhibition for the Compound A+Compound B combination was calculated to be 69% and was statistically significant (p=0.0121). These data suggest that Compound A and Compound B can be safely dosed in a regimen that is effective, supporting further clinical evaluation for the treatment of human gastric cancer.

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described accordingly, all suitable modifications and equivalents may be resorted to, falling within the scope of the present disclosure.

What is claimed is:

1. A method of treating cancer in a subject comprising administering to the subject:
(a) a therapeutically effective amount of at least one cancer stemness inhibitor chosen from cancer stemness inhibitors, prodrugs thereof, pharmaceutically acceptable salts of any of the foregoing, and solvates of any of the foregoing; and
(b) a therapeutically effective amount of at least one kinase-targeting agent chosen from kinase-targeting agents, prodrugs thereof, pharmaceutically acceptable salts of any of the foregoing, or solvates of any of the foregoing.

2. The method according to claim 1, wherein the at least one cancer stemness inhibitor is chosen from STAT3 pathway inhibitors.

3. The method according to claim 1 or 2, wherein the at least one kinase-targeting agent is chosen from kinase inhibitors.

4. The method according to any one of claims 1-3, wherein the at least one cancer stemness inhibitor is chosen from 2-(1-hydroxyethyl)-naphtho[2,3-b]furan-4,9-dione, 2-acetyl-7-chloro-naphtho[2,3-b]furan-4,9-diose, 2-acetyl-7-fluoro-naphtho[2,3-b]furan-4,9-dione, 2-acetylnaphtho[2,3-b]furan-4,9-dione, and 2-ethyl-naphtho[2,3-b]furan-4,9-dione.

5. The method according to any one of claims 1-4, wherein the at least one kinase-targeting agent is chosen from

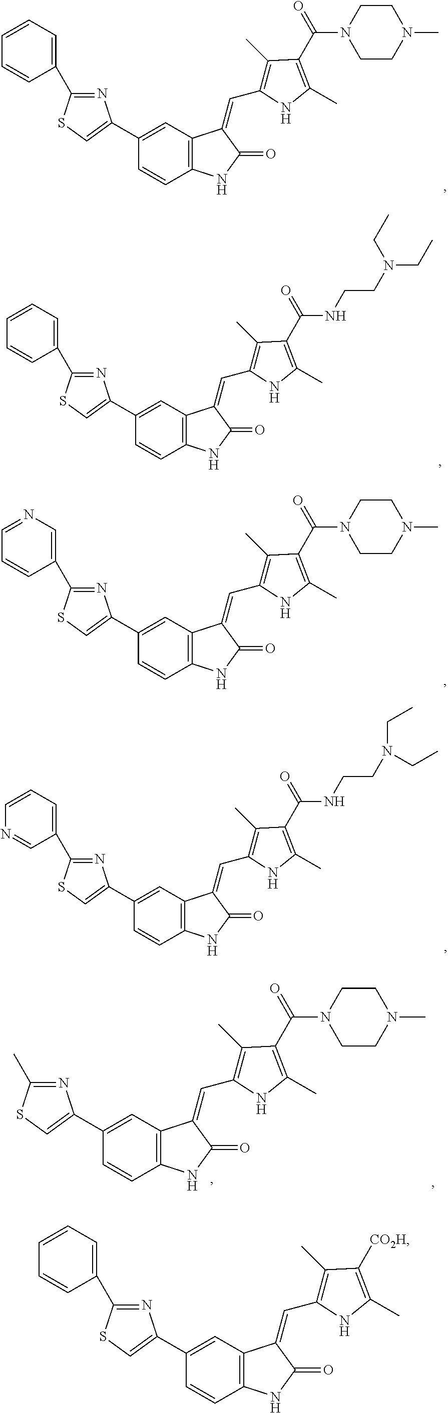

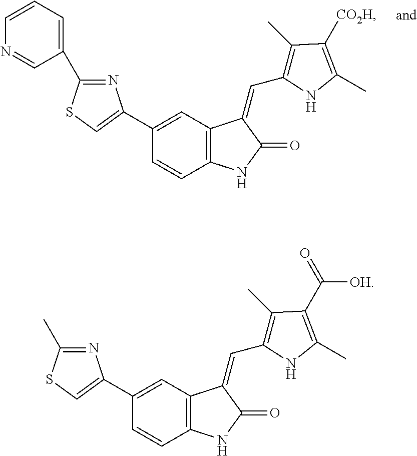

6. A method of treating cancer in a subject, comprising administering to a subject in need thereof:
a therapeutically effective amount of at least one compound of formula A chosen from compounds having formula A: 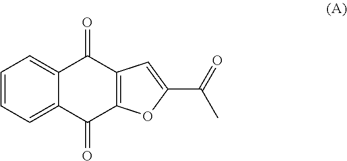 [SMILES: CC(=O)c1cc2c(o1)C(=O)c1ccccc1C2=O]
prodrugs, derivatives, pharmaceutically acceptable salts of any of the foregoing, and solvates of any of the foregoing, and
a therapeutically effective amount of at least one compound of formula B chosen from compounds having formula B: 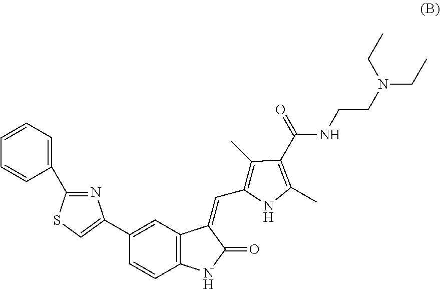 [SMILES: CCN(CC)CCNC(=O)c1c(C)[nH]c(/C=C2\C(=O)Nc3ccc(-c4csc(-c5ccccc5)n4)cc32)c1C]
prodrugs, derivatives, pharmaceutically acceptable salts of any of the foregoing, and solvates of any of the foregoing.

7. A method of inhibiting, reducing, and/or diminishing CSC survival and/or self-renewal, comprising administering:
a therapeutically effective amount of at least one compound of formula A chosen from compounds having formula A: 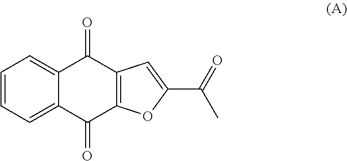 [SMILES: CC(=O)c1cc2c(o1)C(=O)c1ccccc1C2=O]
prodrugs, derivatives, pharmaceutically acceptable salts of any of the foregoing, and solvates of any of the foregoing, and
a therapeutically effective amount of at least one compound of formula B chosen from compounds having formula B: 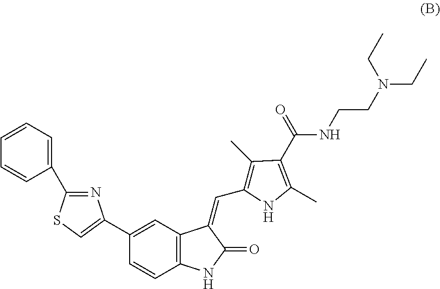 [SMILES: CCN(CC)CCNC(=O)c1c(C)[nH]c(/C=C2\C(=O)Nc3ccc(-c4csc(-c5ccccc5)n4)cc32)c1C]
prodrugs, derivatives, pharmaceutically acceptable salts of any of the foregoing, and solvates of any of the foregoing.

8. A method of treating at least one cancer that is refractory to conventional chemotherapies and/or targeted therapies in a subject, comprising administering:
a therapeutically effective amount of at least one compound of formula A chosen from compounds having formula A: 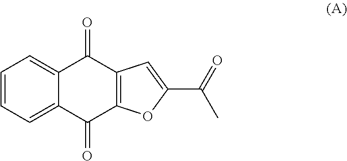 [SMILES: CC(=O)c1cc2c(o1)C(=O)c1ccccc1C2=O]
prodrugs, derivatives, pharmaceutically acceptable salts of any of the foregoing, and solvates of any of the foregoing, and
a therapeutically effective amount of at least one compound of formula B chosen from compounds having formula B: 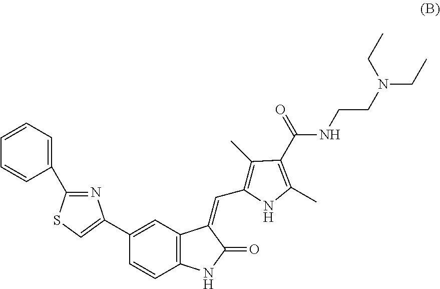 [SMILES: CCN(CC)CCNC(=O)c1c(C)[nH]c(/C=C2\C(=O)Nc3ccc(-c4csc(-c5ccccc5)n4)cc32)c1C]
prodrugs, derivatives, pharmaceutically acceptable salts of any of the foregoing, and solvates of any of the foregoing.

9. A method of preventing cancer relapse in a subject, comprising administering:
a therapeutically effective amount of at least one compound of formula A chosen from compounds having formula A: 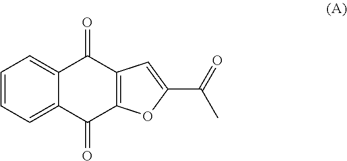 [SMILES: CC(=O)c1cc2c(o1)C(=O)c1ccccc1C2=O]
prodrugs, derivatives, pharmaceutically acceptable salts of any of the foregoing, and solvates of any of the foregoing, and
a therapeutically effective amount of at least one compound of formula B chosen from compounds having formula B: 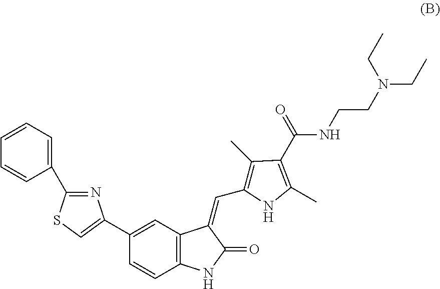 [SMILES: CCN(CC)CCNC(=O)c1c(C)[nH]c(/C=C2\C(=O)Nc3ccc(-c4csc(-c5ccccc5)n4)cc32)c1C]
prodrugs, derivatives, pharmaceutically acceptable salts of any of the foregoing, and solvates of any of the foregoing.

10. A method of suppressing regrowth or recurrent of cancer in a subject, comprising administering:
a therapeutically effective amount of at least one compound of formula A chosen from compounds having formula A: 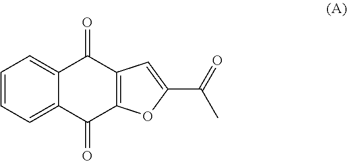 [SMILES: CC(=O)c1cc2c(o1)C(=O)c1ccccc1C2=O]
prodrugs, derivatives, pharmaceutically acceptable salts of any of the foregoing, and solvates of any of the foregoing, and
a therapeutically effective amount of at least one compound of formula B chosen from compounds having formula B: 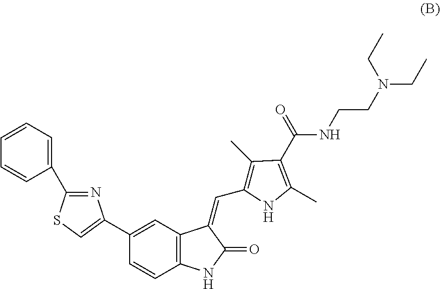 [SMILES: CCN(CC)CCNC(=O)c1c(C)[nH]c(/C=C2\C(=O)Nc3ccc(-c4csc(-c5ccccc5)n4)cc32)c1C]
prodrugs, derivatives, pharmaceutically acceptable salts of any of the foregoing, and solvates of any of the foregoing.

11. The method according to any one of claims 6-10, wherein the at least one compound of formula A is administered orally in an amount ranging from about 80 mg to about 960 mg twice daily.

12. The method according to claim 11, wherein the at least one compound of formula A is administered orally in an amount ranging from about 160 mg to about 240 mg twice daily.

13. The method according to claim 11 or 12, wherein the at least one compound of formula A is administered orally in an amount of about 240 mg twice daily.

14. The method according to any of claims 6-13, wherein the at least one compound of formula B is administered orally in an amount ranging from about 50 mg to about 600 mg once daily.

15. The method according to claim 14, wherein the at least one compound of formula B is administered orally at a dose in an amount ranging from about 100 to about 300 mg once daily.

16. The method according to claim 14, wherein the at least one compound of formula B is administered orally in an amount of about 100 mg daily or about 200 mg daily.

17. The method according to any one of claims 1-16, wherein the cancer is esophageal cancer, gastroesophageal junction cancer, gastroesophageal adenocarcinoma, gastric cancer, chondrosarcoma, colorectal adenocarcinoma, breast cancer, ovarian cancer, head and neck cancer, melanoma, gastric adenocarcinoma, lung cancer, pancreatic cancer, renal cell carcinoma, hepatocellular carcinoma, cervical cancer, brain tumor, multiple myeloma, leukemia, lymphoma, prostate cancer, cholangiocarcinoma, endometrial cancer, small bowel adenocarcinoma, uterine sarcoma, or adrenocorticoid carcinoma.

18. The method according to claim 17, wherein the cancer is unresectable, advanced, refractory, recurrent, or metastatic.

19. A kit comprising (1) at least one compound chosen from compounds having formula A, 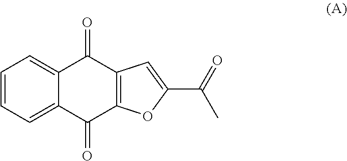 [SMILES: CC(=O)c1cc2c(o1)C(=O)c1ccccc1C2=O]
prodrugs, derivatives, pharmaceutically acceptable salts of any of the foregoing, and solvates of any of the foregoing,
(2) at least one compound chosen from compounds having formula B, 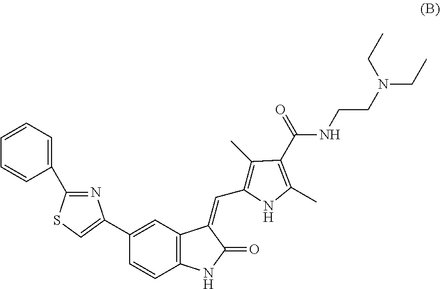 [SMILES: CCN(CC)CCNC(=O)c1c(C)[nH]c(/C=C2\C(=O)Nc3ccc(-c4csc(-c5ccccc5)n4)cc32)c1C]
prodrugs, derivatives, pharmaceutically acceptable salts of any of the foregoing, and solvates of any of the foregoing, and
instructions for administration and/or use.
